(19) United States
(12) Patent Application Publication
(10) Pub. No.: US 2002/0081312 A1
(43) Pub. Date: Jun. 27, 2002

(54) Recombinant cryptosporidium parvum antigen and detection of antibodies thereto

(76) Inventors: Jeffrey W. Priest, Atlanta, GA (US); Patrick J. Lammie, Lawrenceville, GA (US); James P. Kwon, Atlanta, GA (US); Michael J. Arrowood, Duluth, GA (US); Delynn M. Moss, Duluth, GA (US)

Correspondence Address: NEEDLE & ROSENBERG, P.C., The Candler Building, Suite 1200, 127 Peachtree Street, N.E., Atlanta, GA, 30303-1811, US

(21) Appl. No.: 09/734,417

(22) Filed: Dec. 11, 2000

Related U.S. Application Data
(60) Provisional application No. 60/169,797, filed on Dec. 9, 1999.

(51) Int. Cl. A61K 39/002; G01N 33/53; G01N 33/569; C07K 14/44

(52) U.S. Cl. 424/191.1; 530/350; 435/7.3

(57) ABSTRACT

A recombinant protein for the diagnosis and immunization of humans and animals against gastrointestinal disorders of Cryptosporidium parvum (C. parvum) infection contains the essential features of the native antigen and may be used in place of the native antigen. A recombinant protein can be quickly and inexpensively reproduced. A DNA sequence encoding a 17-kDa protein for a surface antigen of C. parvum is also disclosed. The DNA sequence may be inserted into recombinant DNA molecules such as cloning vectors or expression vectors for the transformation of cells and the production of the protein. Test kits for detecting contamination of food or water are also provided. Also disclosed are a detergent extraction method for partial purification of the 17-kDa and 27-kDa surface antigens and ELISA assays employing these proteins for detecting C. parvum antibodies in biological samples.

This application claims priority to U.S. Provisional Application No. 60/169,797 filed Dec. 9, 1999 and U.S. Provisional Application No. 60/174,054 filed Dec. 30, 1999, both of which are herein incorporated by reference.

### FIELD OF THE INVENTION

The present invention relates to the field of immunology and more particularly to a novel 17-kDa protein for eliciting protective immune responses in animals against Cryptosporidium parvum (C. parvum), and recombinant DNA sequences which encode the protein. The invention also relates to a novel method of partially purifying the protein and ELISA methods for detecting antibodies to this antigen and a related 27-(kDa antigen in blood samples.

### BACKGROUND OF THE INVENTION

Cryptosporidiosis is a parasite infection of medical and veterinary importance that affects the epithelial cells of the gastrointestinal, biliary, and respiratory tracts of humans, as well as over 45 different vertebrate species. C. parvum is a food or waterborne protozoan parasite of the intestinal epithelium causing severe intestinal distress. Infection caused by C. parvum is particularly dangerous because it can cause prolonged diarrheal illness that may be potentially fatal for immunocompromised individuals. Since the 1970s, C. parvum has been receiving increased world wide attention as the frequency of outbreaks and the number of individuals infected increase across the globe. For example, in an outbreak reported in Milwaukee, Wis. in 1993, approximately 400,000 people were infected with C. parvum. Therefore, the U.S. Environmental Protection Agency has begun the process of mandating that waters in the United States be tested for Cryptosporidium parasites.

Although cryptosporidiosis occurs worldwide, children, travelers to foreign countries, male homosexuals, and medical personnel caring for patients with the disease are at particular risk. Apart from humans, Cryptosporidium infections are widespread in several other vertebrates including mammals, reptiles and fish. Accordingly, the frequency of cryptosporidiosis in animal handlers and veterinarian personnel is reported to be relatively high.

As C. parvum organisms invade the surface of intestinal cells, the host experiences symptoms such as reduced appetite, severe diarrhea, abdominal cramping, and chronic fluid loss. The symptoms generally persist for five to eleven days, and then rapidly abate. However, in immunocompromised individuals, such as malnourished children, individuals with congenital hypogammaglobulinemia, those receiving immunosuppressants for cancer therapy or organ transplantation, and patients with AIDS, onset of the disease is more gradual and diarrhea is more severe, causing extreme fluid losses. Unless the underlying immunologic defect is corrected, the diarrhea may continue persistently or remittently for life because there is no effective, specific anti-C. parvum therapy available at present. Although some patients have responded positively to therapy with conventional antibiotics such as spiramycin and paromomycin, the result of infection is frequently fatal for immunocompromised individuals. In fact, cryptosporidiosis has been reported as one of the predominant causes of death in immunocompromised patients.

The organism poses a significant public health threat because of the resistance of oocysts to chlorine treatment and because of the large number of unfiltered surface water supplies in the United States. The magnitude of this threat has been difficult to quantify because of the poor sensitivity of stool-based assays for C. parvum oocysts and because of the intermittent nature of oocyst shedding by infected persons. Therefore, a quantification method for providing a realistic estimate of the number of persons infected each year is needed.

In the past, serum antibody responses to C. parvum infection have been tracked using a crude extract of disrupted oocysts as antigen with either an enzyme-linked immunoassay (ELISA) as taught by Ungar et al., J. Infect. Dis. 153:570-578 (1986), or a Western blot assay (Moss et al., J. Euk. Microbiol. 41:52S-55S (1994), Moss et al., Am. J. Trop. Med. Hyg. 58:110-118 (1998), Ungar and Nash, Infect. Immun. 53:124-128 (1986)). Recent work by Moss et al. has shown that the crude oocyst antigen ELISA is not a sensitive means of detecting antibodies against the immunodominant low molecular weight C. parvum antigens (Moss et al., Am. J. Trop. Med. Hyg. 58:110-118 (1998), Moss et al., J Infect. Dis. 178:827-833 (1998)). Although the Western blot assay is quite sensitive, it suffers from a limited linear range, and the antibody levels are difficult to quantitate by densitometry. A Western blot assay is also technically challenging and labor intensive in that a gradient sodium dodecyl sulfate (SDS) polyacrylamide gel is required for optimal antigen separation. An assay capable of high sample throughput and easy quantitation is required for planned population-based studies of the risk factors for C. parvum infection in both immunocompetent and immunocompromised persons.

In light of the potentially fatal consequences of C. parvum infection, its resistance to chlorine treatment, and the large number of unfiltered water supplies, means for studying past infection in populations are necessary. Estimates of the number of persons infected with C. parvum each year would also be facilitated by the development of sensitive and specific assays lacking the drawbacks of the Western blot assay. Therefore, an efficient assay for the detection of human serum IgG antibodies against C. parvum is needed.

Much expense and time are required for passaging Cryptosporidium in calves and purifying oocysts for antigen extraction. Therefore, what is also needed is an inexpensive means for producing sufficient quantities of the antigen for epidemiologic studies, for studies of the role of the humoral response in immunity, research analysis, for use in immunoassays and for producing vaccines and therapeutic drugs to prevent and treat C. parvum infections.

Considerable efforts have been made to develop and improve C. parvum immunization methodologies. One such C. parvum immunization attempt includes U.S. Pat. No. 5,106,618 to Beck et al., which describes treatment of gastrointestinal disorders of parasitic protozoan and bacterial origin by drinking milk having a concentrated amount of antibodies to the mixture of bacterial strains and species shown in Table 1. However, there was no immunization with a purified, recombinant Cryptosporidium antigen. Successful immunization with the recombinant protein may be prophylactic and could eliminate the necessity of passive transfer after an individual has been infected. Furthermore, immunization with a single recombinant C. parvum protein could have fewer side effects than immunization with a heat-killed mixture of 26 bacterial species and strains.

U.S. Pat. No. 5,591,434 to Jenkins et al. discloses recombinant proteins useful for the production of hyperimmune colostrum that may be used to confer passive immunity against C. parvum. However, these proteins do not bind to antibodies found in sera of previously and currently infected human patients.

For the immunocompromised host, the need for efficacious therapy is more pronounced than for the immunocompetent individual. A vast array of antimicrobial, immunomodulatory and nonspecific anti-diarrheal drugs, as well as special diets, have been administered to such patients. With few exceptions, attempts at therapeutic intervention have met with failure, both in the control of enteric symptoms and in the eradication of the parasite.

An important need exists, therefore, for an effective method for both preventing and treating disorders due to Cryptosporidia.

What is also needed are methods and compositions for immunization against C. parvum infection.

In summary, existing detection methods and immunizations for C. parvum parasites are ineffective. Antigens and immunologic detection methods utilizing the antigens are essential. Useful immunizations against disease-parasites are required for safeguarding against C. parvum. What is needed, therefore, are antigenic proteins, and the sequences which encode them, that are effective for the detection of C. parvum and for the immunization of animals against cryptosporidiosis and inexpensive methods for the production of the proteins.

### SUMMARY OF THE INVENTION

Disclosed are a recombinant form of a 17-kDa antigen of C. parvum (referred to herein as recombinant Cp17) and nucleic acid and amino acid sequences encoding the antigenic protein. The recombinant 17-kDa antigen binds to antibodies found in sera of previously and currently infected patients and to a known monoclonal antibody to the native 17-kDa antigen. The 17-kDa antigen of the invention has a carboxy-terminal signal sequence for the addition of a glycosylphosphotidylinositol (GPI) membrane anchor and an amino terminus sequence for targeting to the endoplasmic reticulum.

The recombinant form of the antigen described herein is preferred over the native antigen because the protein produced by this composite DNA contains the essential features of the native antigen and can be quickly and inexpensively produced. The isolation or extraction of native antigen from C. parvum, on the other hand, is expensive and time consuming to produce. Cryptosporidium must be passaged in calves and the oocysts purified for antigen extraction. Therefore, it is seen that the recombinant protein of the present invention may be used interchangeably with the native form, while also saving large amounts of time and money during production.

Uses for the recombinant Cp17 protein include scientific or diagnostic analysis of C. parvum and a target for vaccine/drug development. The recombinant protein is useful as a reagent in assays, such as immunoassays, and for the diagnosis or monitoring of C. parvum infection in a patient sample. Preferably, the antigenic protein binds to antibodies present in the biological sample being tested. The detection of antigenic proteins bound to antibodies indicates that the human or animal from whom the biological sample was taken is or was infected with C. parvum.

The recombinant protein is also useful for the construction of pharmaceutical compositions, such as vaccines, comprising an immunogenic amount of the antigenic protein and a pharmaceutically acceptable carrier. The pharmaceutical compositions are administered to humans or animals to prevent, minimize or reduce C. parvum infection, particularly to patients who have been exposed to C. parvum or are immunocompromised or to individuals, such as infant daycare workers or animal handlers, who are more likely to become exposed to C. parvum infection.

The recombinant antigenic protein is additionally useful for the generation of antibodies, both monoclonal and polyclonal, that are reactive with and can be used to detect C. parvum proteins in a sample. Such antibodies are particularly useful for laboratory research purposes to study C. parvum.

Kits for the detection of C. parvum in a biological sample are also provided. The kits contain the recombinant antigenic protein. The kits may optionally contain an apparatus and one or more containers for obtaining and storing the sample prior to and during analysis and suitable buffers and other reagents to facilitate antibody-antigen binding and detection. Each component of each kit may be provided in separate containers or any combination of the components may be provided in a single container. The nucleic acid molecules encoding the antigenic protein are useful for the production thereof by recombinant means.

Methods for making the recombinant Cp17 antigen include methods known in the art and those described in the appended Examples. Proteins, peptides, and polypeptides of the invention may be produced by various methods, for example, they may be chemically or enzymatically synthesized, may be produced by recombinant DNA methods, may be prepared from the Cp17 antigen, or may be prepared from standard oocyst extraction.

The methods used herein include partially purifying sufficient quantities of the target antigen from oocysts using a modification of the standard Triton X-114 extraction protocol of Bordier J. Biol. Chem. 256:1604-1607 (1981) described in Example 1. This new Triton X-114 protocol was specifically designed to enhance the purification of GPI anchored proteins (Ko and Thompson, Anal. Biochem. 224:166-172 (1995)) extracted from sonicated whole oocysts.

In a preferred embodiment, the C. parvum detection methods are enzyme-linked immunoassays (ELISAs) for the detection and quantitation of serum IgG antibodies against one or more of the native 17-kDa antigen, the recombinant 17-kDa antigen and a 27-kDa antigen. The assays utilize a recombinant form of the 27-kDa antigen and a partially purified native fraction isolated from sonicated whole oocysts that contains the 17-kDa antigen.

Positive responses with the recombinant 27-kDa antigen ELISA showed a sensitivity and specificity of 90% and 92%, respectively, when correlated with a reference standard immunoblot seroassay. Similarly, positive responses with the partially purified native 17-kDa antigen ELISA correlated with the immunoblot results for the 17-kDa antigen with a sensitivity and specificity of 90% and 94%, respectively. For both ELISAs the median IgG antibody levels for serum sets collected during waterborne outbreaks of C. parvum were at least 2.5-fold higher than the levels determined for a non-outbreak set. In comparisons with the immunoblot, the new ELISAs were more specific and, for the 27-kDa antigen ELISA, more sensitive than the crude oocyst antigen ELISA currently in use.

Uses for the ELISA methods include studying past infections of animal populations, characterizing the human antibody response to infection by C. parvum and estimating the number of persons previously infected by C. parvum.

Accordingly, it is an object of the present invention to provide recombinant proteins that contain the essential features of the native antigen, and the genes which encode them, that are effective for the detection of C. parvum antibodies and the immunization of animals and humans against C. parvum oocysts.

It is yet another object of the present invention to provide a recombinant 17-kDa antigenic protein of C. parvum that is fast, easy and inexpensive to produce.

It is yet another object of the present invention to provide methods and compositions useful for protecting animals against C. parvum.

It is yet another object of the present invention to provide methods for detecting C. parvum in a biological sample, wherein the methods have high specificity for C. parvum.

It is yet another object of the present invention to provide methods for detecting C. parvum that are rapid, simple, reliable, sensitive, and not labor intensive.

It is yet another object of the present invention to provide an antigen in an ELISA format to characterize the human antibody responses that develop following C. parvum infection.

These and other objects, features and advantages of the present invention will become apparent after a review of the following detailed description of the disclosed embodiments and the appended claims.

### BRIEF DESCRIPTION OF THE FIGURES

FIG. 1. Nucleotide sequence of the gene coding for the recombinant C. parvum 17-kDa antigen (recombinant Cp17) (SEQ ID NO:1) and deduced amino acid sequence (SEQ ID NO:2) The deduced amino acids are listed above the first base of each codon in the DNA sequence, and two in-frame stop codons are indicated by pound (#) signs. Two potential methionine initiation codons are indicated in bold and italicized in the DNA sequence. Positions of various deoxyoligonucleotide probes and primers are indicated by underlines. The cleavage site for an amino-terminal signal peptide predicted using the SignalP program of Nielsen et al; Prot. Eng. 10, 1-6 (1997) is indicated by an arrow. The amino acid sequence of the putative mature recombinant Cp17 protein is indicated by bold letters, and the recombinant Cp17 fragment protein sequence is enclosed in parentheses. The mature amino terminus is circled. A stretch of 20 hydrophobic residues at the carboxy terminus of the protein sequence is indicated in italics. A region that includes six small amino acids and that may contain an ω site for GPI anchor attachment is enclosed in brackets.

FIG. 2 compares the predicted amino acid sequence of recombinant Cp17 cDNA and the previously published Khromatov sequence.

FIG. 3 shows graphical representations of the changes in antibody levels in paired serum samples from symptomatic individuals as measured by the two ELISAs described herein.

### DETAILED DESCRIPTION

Disclosed are a recombinant 17-kDa antigen of C. parvum (recombinant Cp17) and an isolated nucleic acid molecule encoding the antigenic protein. The amino acid sequence of the recombinant Cp17 protein is also disclosed. The recombinant 17-kDa antigen binds to antibodies found in sera of previously and currently infected patients and to a known monoclonal antibody of the native Cp17 (C6C1, Arrowood, Ph.D. thesis, University of Arizon, Tuscon, Ariz. (1988)). The recombinant 17-kDa antigen has a carboxy-terminal signal sequence for the addition of a GPI membrane anchor and an amino terminus sequence for targeting the endoplasmic reticulum.

The recombinant form of the antigen described herein is preferred over the native antigen because the protein produced from the DNA contains the essential features of the native antigen but can be quickly and inexpensively produced. The isolation or extraction of native antigen from C. parvum, on the other hand, is expensive and time consuming because the Cryptosporidium must be passaged in calves and the oocysts collected and purified prior to antigen extraction. Therefore, it is seen that the recombinant protein of the present invention may be used in place of the native form at a reduced cost.

Uses for the recombinant Cp17 protein include scientific or diagnostic analysis of C. parvum and a target for vaccine or drug development. The recombinant protein is useful as a reagent in assays, such as immunoassays, for the diagnosis or monitoring of C. parvum infection in a patient sample. Preferably, the antigenic protein binds to antibodies present in the biological sample being tested. The detection of antigenic proteins bound to antibodies indicates that the human or animal from which the biological sample was taken is currently infected with C. parvum or has been infected in the past. The ability to gather this type of data makes antibodies against the 27-kDa and 17-kDa antigens useful markers for past infection in population-based studies of the risk factors associated with Cryptosporidium infection. In most cases, the cryptosporidiosis has resolved spontaneously by the time IgG antibodies are detected in the serum. Current infections are likely to be detected only if they are chronic.

As described below in Example 9, the present invention also provides an isolated DNA sequence which encodes an antigenic protein effective for eliciting antibody production in an animal or human against C. parvum.

The complete recombinant Cp17 open reading frame that encodes the peptide sequences was cloned and sequenced, and the recombinant mature protein fragment expressed and assayed for reactivity against both sera from cryptosporidiosis patients and a known monoclonal antibody to the native Cp17 (C6C1; Arrowood, Ph.D. thesis, University of Arizona, Tuscon, Ariz. (1988)). The chemical and enzymatic characteristics of the native Cp17 are consistent with a GPI-anchored antigen yet no previous researcher has isolated a sequence to code for a Cp17 protein with a GPI-anchor site.

### Recombinant C. parvum Antigen

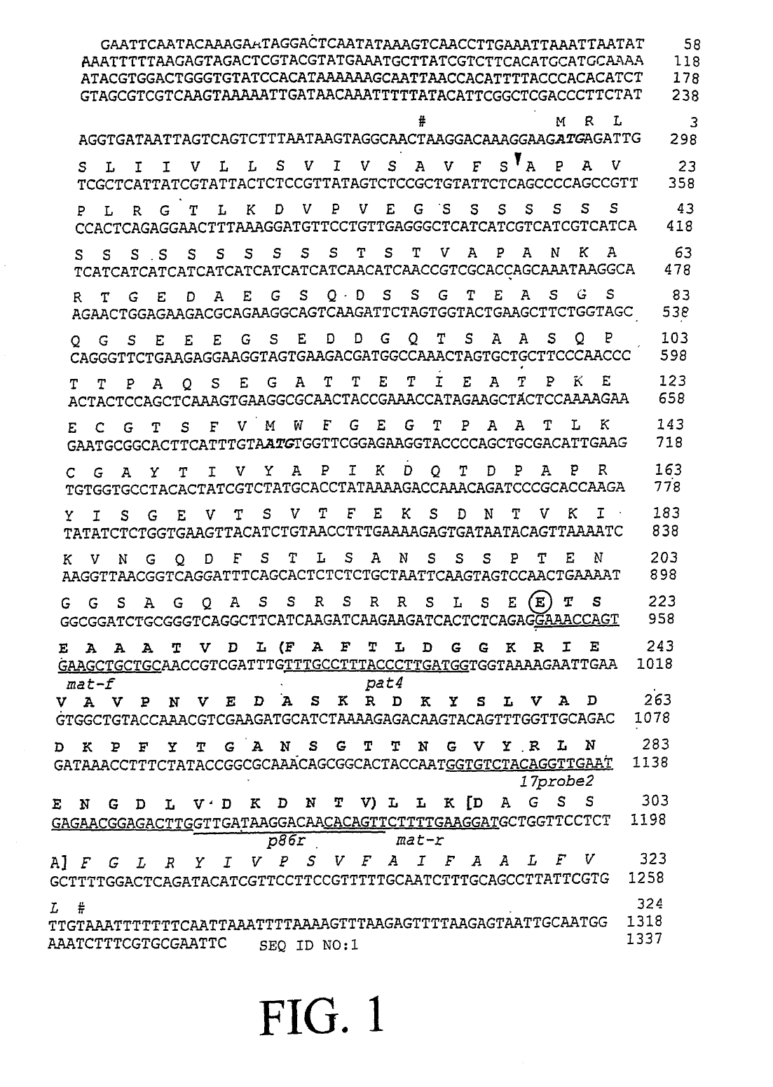

The invention encompasses cDNA clones having a nucleotide sequence (SEQ ID NO:1) encoding a immunodominant 17-kDa C. parvum surface antigen protein (recombinant Cp17), the deduced amino acid sequence (SEQ ID NO:2), as well as DNA sequences which encode proteins having amino acid sequences that are homologous to that of FIG. 1. “Homologous” proteins are defined herein as proteins having an amino acid sequence sufficiently duplicative of recombinant Cp17 protein to be antigenic and capable of eliciting antibody production against C. parvum. DNA sequences encoding recombinant Cp17 protein with the amino acid sequence shown in FIG. 1, and DNA sequences which encode homologous proteins and which also hybridized in the DNA sequence of FIG. 1 (or its complement) under stringent conditions are particularly preferred. The GenBank accession number of the nucleotide sequence shown in FIG. 1 is AF114166.

The recombinant antigenic proteins of the present invention and antibodies thereto are useful as reagents in assays for the detection of C. parvum infection in a biological sample, such as a patient sample. The nucleic acid molecules encoding the recombinant antigenic proteins are useful for the production of the recombinant antigenic proteins and as probes for detecting C. parvum nucleic acids.

The appended examples describe partial purification and sequencing of native immunodominant 17-kDa surface antigen from sporozoites, and cloning of a 975 bp open reading frame from C. parvum that includes all of the 17-kDa antigen peptide sequences.

### Definitions

The terms “a”, “an” and “the” as used herein are defined to mean one or more and include the plural unless the context is inappropriate.

“Peptides,” “polypeptides” and “oligopeptides” are chains of amino acids (typically L-amino acids) whose alpha carbons are linked through peptide bonds formed by a condensation reaction between the carboxyl group of the alpha carbon of one amino acid and the amino group of the alpha carbon of another amino acid. The terminal amino acid at one end of the chain (i.e., the amino terminal) has a free amino group, while the terminal amino acid at the other end of the chain (i.e., the carboxy terminal) has a free carboxyl group. As such, the term “amino terminus” (abbreviated N-terminus) refers to the free alpha-amino group on the amino acid at the amino terminal of the peptide, or to the alpha-amino group (imino group when participating in a peptide bond) of an amino acid at any other location within the peptide. Similarly, the term “carboxy terminus” (abbreviated C-terminus) refers to the free carboxyl group on the amino acid at the carboxy terminus of a peptide, or to the carboxyl group of an amino acid at any other location within the peptide.

Typically, the amino acids making up a peptide are numbered in order, starting at the amino terminal and increasing in the direction toward the carboxy terminal of the peptide. Thus, when one amino acid is said to “follow” another, that amino acid is positioned closer to the carboxy terminal of the peptide than the preceding amino acid.

The term “residue” is used herein to refer to an amino acid that is incorporated into a peptide by an amide bond. As such, the amino acid may be a naturally occurring amino acid or, unless otherwise limited, may encompass known analogs of natural amino acids that function in a manner similar to the naturally occurring amino acids (i.e., amino acid mimetics). Moreover, an amide bond mimetic includes peptide backbone modifications well known to those skilled in the art.

The phrase “consisting essentially of” is used herein to exclude any elements that would substantially alter the essential properties of the proteins to which the phrase refers. Thus, the description of a peptide “consisting essentially of . . . ” excludes any amino acid substitutions, additions, or deletions that would substantially alter the biological activity of that peptide.

The word “antigen”, as used herein, refers to an entity or fragment thereof which can induce an immune response in a mammal. The term includes immunogens and regions responsible for antigenicity or antigenic determinants.

The term “antigenic determinant” or “antigenic epitope” refers to a peptide or region of a C. parvum protein recognized by an antibody, e.g., in serum raised against wild-type C. parvum.

The phrases “specifically binds to a peptide” or “specifically immunoreactive with”, when referring to an antibody, refer to a binding reaction which is determinative of the presence of a peptide in the presence of a heterogeneous population of proteins and other biological substances. Under designated immunoassay conditions, the specified antibodies bind preferentially to a particular peptide and do not bind in a significant amount to other proteins present in the sample. Specific binding to a peptide under such conditions requires an antibody that is selected for its specificity for a particular protein. A variety of immunoassay formats may be used to select antibodies specifically immunoreactive with a particular protein. For example, solid phase ELISA immunoassays are routinely used to select monoclonal antibodies specifically immunoreactive with a protein.

The terms “nucleic acid” or “nucleic acid molecule”, as used herein, refer to a deoxyribonucleotide (DNA) or ribonucleotide (RNA) in either single- or double-stranded form, and unless otherwise limited, encompass known analogs of natural nucleotides which can function in a manner similar to the naturally occurring nucleotides.

The phrase “nucleic acid sequence encoding” refers to a nucleic acid sequence which directs the expression of a specific protein or peptide. The nucleic acid sequence includes both the DNA sequence that is transcribed into RNA and the RNA sequence that is translated into the protein. The nucleic acid sequence includes both the full length nucleic acid sequence as well as any non-full length sequences derived from the full length sequence. It will be understood by those of skill that the sequence includes the degenerate codons of the native sequence or sequences which may be introduced to provide codon preference in a specific host cell.

The phrase “conservatively modified variations” of a particular nucleic acid sequence refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given peptide. Such nucleic acid variations are silent variations, which are one species of conservatively modified variations. One of skill will recognize that each codon in a nucleic acid (except AUG, which is ordinarily the only codon for methionine) can be modified to yield a functionally identical molecule by standard techniques. Accordingly, each silent variation of a nucleic acid which encodes a peptide is implicit in any described amino acid sequence and are hence encompassed by the instant invention.

Furthermore, one of skill will recognize that, as mentioned above, individual substitutions, deletions or additions which alter, add or delete a single amino acid or a small percentage of amino acids (typically less than 5%, more typically less than 1%) in an encoded sequence are conservatively modified variations where the alterations result in the substitution of an amino acid with a chemically similar amino acid. Conservative substitution tables providing functionally similar amino acids are well known in the art. The following six groups each contain amino acids that are conservative substitutions for one another:

1) Alanine (A), Serine (S), Threonine (T);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V); and

6) Phenylalanine (F), Tyrosine (Y), Tryptophan (W).

Two polynucleotides or polypeptides are said to be “identical” if the sequence of nucleotides or amino acid residues in the two sequences is the same when aligned for maximum correspondence. Optimal alignment of sequences for comparison may be conducted by the local homology algorithm of Smith and Waterman Adv. Appl. Math. 2:482 (1981), by the homology alignment algorithm of Needleman and Wunsch J Mol. Biol. 48:443 (1970), by the search for similarity method of Pearson and Lipman Proc. Natl. Acad. Sci. (U.S.A.) 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection. These references are incorporated herein by reference.

The term “substantial identity” means that a polypeptide comprises a sequence that has at least 80% sequence identity, preferably 90%, more preferably 95% or more, compared to a reference sequence over a comparison window of about 10 to about 20. Another indication that polypeptide sequences are substantially identical is if one peptide is immunologically reactive with antibodies raised against the other peptide. Thus, the proteins described herein include peptides immunologically reactive with antibodies raised against the disclosed proteins.

The phrases “isolated” or “biologically pure” refer to material which is substantially or essentially free from components which normally accompany it as found in its native state. Thus, the proteins described herein do not contain materials normally associated with their in situ environment, e.g., other proteins from a sporozoite membrane. Typically, the isolated, immunogenic C. parvum proteins described herein are at least about 80% pure, usually at least about 90%, and preferably at least about 95% as measured by band intensity on a silver stained gel.

Protein purity or homogeneity may be indicated by a number of methods well known in the art, such as polyacrylamide gel electrophoresis of a protein sample, followed by visualization upon staining. For certain purposes, high resolution will be needed and HPLC or a similar means for purification utilized.

The term “residue” refers to an amino acid (D or L) or an amino acid mimetic incorporated in a oligopeptide by an amide bond or amide bond mimetic. An amide bond mimetic includes peptide backbone modifications well known to those skilled in the art.

The term “treating” as used herein means that the symptoms of the disorder and/or pathogenic origin of the disorder be ameliorated or completely eliminated.

The term “administer” as used herein means any method of treating a subject with a substance, such as orally, intranasally, parenterally (intravenously, intramuscularly, or subcutaneously) or rectally.

DNA sequences which are “substantially homologous” to the nucleotide sequence of FIG. 1 are also encompassed by the invention. As defined herein, two DNA sequences are substantially homologous when at least 85% (preferably at least 90% and most preferably 95%) of the nucleotides match over the defined length of the sequence. Sequences that are substantially homologous can be identified in a Southern hybridization experiment under stringent conditions as is known in the art. See, for example, Sambrook et al., MOLECULAR CLONING: A LABORATORY MANUAL, second edition, Cold Spring Harbor Laboratory Press, New York (1989) or DNA Cloning: A Practical Approach, Vol. I and II (Ed. D. N. Glover), IRL Press, Oxford, 1985 (both of which are incorporated by reference herein). The DNA sequences of the invention can be used to prepare recombinant DNA molecules by cloning in any suitable vector.

### Comparison to Known Antigen Sequences

Several groups have previously reported cloning Cryptosporidium antigens in the 17-kDa size range. However, none of these proteins have a carboxy-terminal signal sequence (a short stretch of approximately 15-25 hydrophobic residues) that would be consistent with the addition of a GPI membrane anchor, nor do they have the hydrophobic characteristics of a membrane associated protein that would be extracted by detergent. Because proteins can be either transmembrane (hydrophobic) or GPI-anchored, the reported proteins not consistent with the known characteristics of the native 17-kDa antigen.

Furthermore, the antigens that have been previously reported are not immunoreactive with known C. parvum monoclonal antibodies or those found in sera. Because an antigen is defined by the antibody reactivity, it is believed that the reported proteins discussed below are not functionally equivalent to native 17-kDa C. parvum antigens.

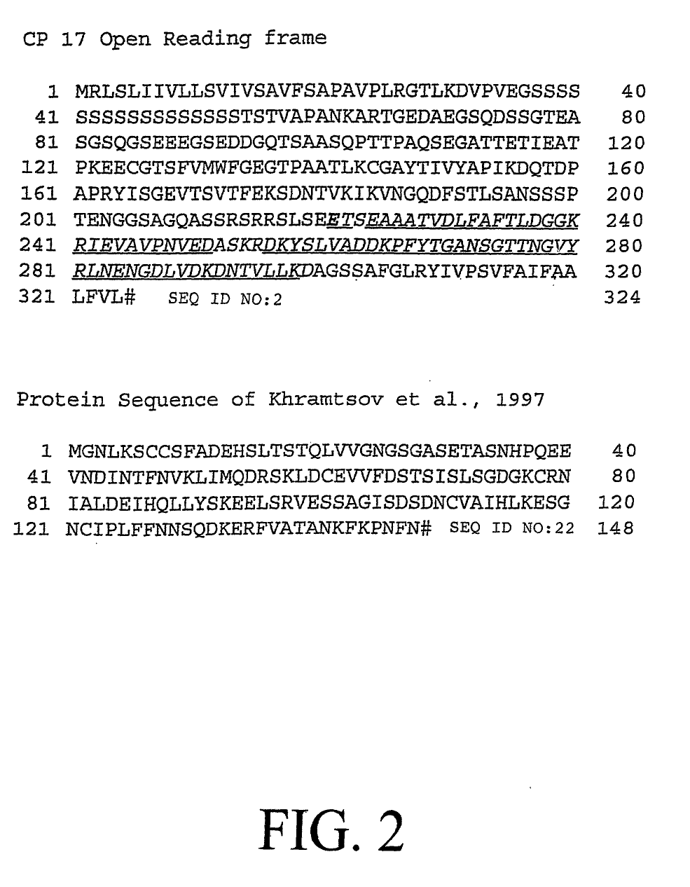

Jenkins and Fayer, et al., Mol. Biochem. Parasitol 71:149-152 (1995) reported the cloning of the 14-kDa C. parvum S15 ribosomal protein using an antiserum against total unfractionated C. parvum antigens. Jenkins et al., Infec. and Immun. 61:2377-2382 (1993) (U.S. Pat. No. 5,591,434) and Khramtsov et al., Res. Commun. 230:164-166 (1997) reported the cloning of the immunodominant 15-kDa surface antigen using a monoclonal antibody (5C3) that recognizes a surface glycoprotein. Although both groups reported having expressed the recombinant antigens, neither reported having tested the antigens with serum from human cryptosporidiosis patients. A comparison of the Khramtsov protein sequence (SEQ ID NO:22) and the open reading frame of the present invention is seen in FIG. 2.

Mead et al. (J. Euk. Microbiol. 41, 51S (1994)) reported cloning an antigen that reacted with monoclonal antibody C6C 1, a monoclonal antibody that recognizes both the native 17-kDa antigen and EF1α (Arrowood, Ph.D. thesis, University of Arizona, Tuscon, Ariz. (1988); Bonafonte et al., Biochim. Biophys. Acta. 1351:256-260 (1997)).

The present inventors expressed the Khramstov et al., protein in a recombinant system that worked well for the 27-kDa antigen (Perryman et al., Mol. Biochem. Parasitol. 80:137-147 (1996)). However, consistent reactivity against this protein using a large panel of banked sera from cryptosporidiosis patients and monoclonal antibodies could not be shown. It is assumed that either the expressed proteins lacked an epitope essential for recognition by the antibodies present in human serum or the native immunodominant 17-kDa antigen was not represented among the proteins. This work is summarized in Table 1.

The deduced protein sequence of FIG. 1, on the other hand, contains a carboxy-terminal signal sequence for the addition of a GPI membrane anchor which is not present in the sequences provided by Jenkins and Khramtsov, Mead, and Jenkins and Fayer. (Jenkins et al., Infec. and Immun. 61:2377-2382 (1993); Khramtsov et al., Res Commun. 230:164-166 (1997); Mead et al., J. Euk. Microbial. 41, 51S (1994); Jenkins and Fayer, et al., Mol. Biochem. Parasitol. 71:149-152 (1995)). Such an anchor is shown to be on the native 17-kDa antigen in Examples 6 and 7. Immunologic identity is shown between a recombinant protein of the present invention and the native 17-kDa antigen which is not found with the proteins of Jenkins and Khramtsov, Mead, and Jenkins and Fayer referenced above.

There are no similarities between the Jenkins et al. sequence and the Cp17 sequence.

### Comparison of Recombinant Cp17 to the Native Cp17 Antigen

The recombinant protein does not have a GPI anchor, and is not modified at the senne-223 or threonine-228 and threonine-235. The recombinant protein can be produced either as the full-length open reading frame product, as the mature protein coding sequence, or as the mature fragment. Additionally, the mature recombinant protein has a lower molecular weight than the native antigen, as shown in Example 5.

### Immunization and Vaccine Studies

The recombinant 17-kDa antigen described herein may be used to prepare therapeutic drugs and immunizations for humans or animals. The 17-kDa antigen may play an important role in host cell epithelial recognition or invasion because it has characteristics similar to other proteins that are involved in host cell recognition and invasion. These other characteristics include: the nativel7-kDa antigen is similar in apparent molecular weight to the target of the 11A5 antibody of Gut and Nelson, (J. Euk. Microbiol. 41:42S-43S (1994)) is present in sporozoite glide trails, is localized to the sporozoite surface (Arrowood, Ph.D. thesis, University of Arizona, Tuscon, Ariz. (1988)), and binds the same GalNAc-specific lectins as the 11A5 target (Jacalin, Vicia villosa lectin (VVL), Helix pomatia lectin (HPA), and Wistaria floribunda lectin (WFA)).

Given that the antibodies in human serum recognize the peptide component of the recombinant Cp17 antigen, that the recombinant antigen is stage specific for those cells that recognize and penetrate epithelial host cells, and that antibodies found in human sera that bind the recombinant antigen correlate with protection from symptomatic infection (Moss, et al., J. Infect. Dis. 178:827-833 (1998)), the recombinant Cp17 antigen described herein is useful as a vaccine.

A vaccine for immunization of a human or animal host comprises administration to a human or animal an immunogenic amount of a C. parvum peptide or antigen, or DNA or RNA encoding the peptide or antigen, capable of evoking production of anti-C. parvum antibodies. The vaccine evokes in the human or animal an active immunity against C. parvum infection.

In a preferred embodiment, the vaccine contains an appropriate pharmaceutically acceptable adjuvant, such as those well known in the art. Adjuvants may include, but are not limited to incomplete Freund's adjuvant (IFA), Freund's complete adjuvant, saponins, Quil A, mineral oil, aluminum hydroxide, aluminum phosphate, muramyl dipeptide, block copolymers and synthetic polynucleotides.

In another preferred embodiment, the protein or a fragment thereof is incorporated into a vaccine with a suitable carrier and suitable doses of the vaccine administered to the non-immune animal or human.

The vaccine may be administered by any of a number of well-known methods. For example, the administration may involve subcutaneous, intradermal, or intramuscular injection. Suitable carriers include buffers and saline solutions appropriate for the animal or human. Alternatively, administration may be oral, such as by a capsule.

In addition, the carrier may also contain a preservative, such as a bacteriostat and/or fungistat. The carrier may also contain an immunopotentiator.

Additional information on carriers, adjuvants and methods of administration may be found in U.S. Pat. No. 5,028,694, U.S. Pat. No. 5,858,378, and WO 98/06430, each of which is herein incorporated by reference.

### Methods for Making the Recombinant Cp17 Antigen

The recombinant 17-kDa antigen is produced by inserting the mature protein coding sequence set forth below the bold amino acids in FIG. 1 (Nucleotides 950-1186 of SEQ ID NO:1) into an expression vector and expressing the protein in accordance with methods known in the art. See, for example, Sambrook et al., MOLECULAR CLONING: A LABORATORY MANUAL, second edition, Cold Spring Harbor Laboratory Press, New York (1989) or DNA Cloning: A Practical Approach, Vol. I and II (Ed. D. N. Glover), IRL Press, Oxford (1985).

Purified antigens can also be prepared from C. parvum oocysts using the modified Triton X-114 purification method described herein. Preferred features of the new Triton X-114 extraction protocol include solubilizing antigens by protein content rather than cell number; modifying the extraction buffer to 20 mM HEPES rather than 10 mM Tris and including PMSF and EDTA; performing extractions for 30 minutes at 4° C. rather than 1 hour at 0° C.; centrifuging at 12000 g for 15 minutes rather than 8800 g for 10 minutes; phase partitioning at 37° C. for 10 minutes rather than 32° C. for 12 minutes; and employing 20 mM HEPES as resuspension buffer rather than 10 mM Tris. Example 1 particularly describes the modified Triton X-114 extraction protocol.

### Detection of Antibodies to recombinant Cp17 and Cp27 Antigens

The recombinant Cp17 antigen described herein can be used in an ELISA format to characterize the human antibody responses that develop after C. parvum infection and to conduct in vitro and in vivo studies on the ability of antibodies to block infection. Preferably, only the mature recombinant protein and the mature fragment are used in the antibody assays.

Most susceptible species of animals, including humans, develop a serum IgG antibody response to two immuno dominant C. parvum sporozoite surface antigens in the 17-kDa (Cp17) and 27-kDa size ranges (Moss et al., J. Euk. Microbiol. 41:52S-55S (1994), Moss et al., Am. J. Trop. Med. Hyg. 58:110-118 (1998), Moss et al., J. Infect. Dis. 178:827-833 (1998); Mead et al., J. Parasitol. 74, 135-143 (1988); Ortega-Mora et al., Vet. Parasitol. 53:159-166 (1994); Peeters et al., Infect. Immun. 60:2309-2316 (1992); Reperant et al., FEMS. Microbiol. Lett. 99, 7-14 (1992); Reperant et al., Vet. Parasitol. 55, 1-13 (1994)). In human volunteer studies (DuPont et al., N. Engl. J. Med. 332:855-859 (1995)), the presence of an IgG antibody to this antigen has been associated with protection from diarrheal symptoms (Moss et al., J. Infect. Dis. 178:827-833 (1998)).

Immunoassays are used to detect and quantify biological and chemical analytes in a sample. Immunoassays are based on the highly specific binding reaction between an antibody, or analyte receptor, and an antigen recognized by the antibody or antigen receptor. Antibodies are binding proteins produced by the immune system of vertebrates in response to substances identified by the immune system as foreign. Immunoassays are commonly used by the medical community to determine the presence, amount or identity of analyte in a biological sample for purposes such as diagnosis and for monitoring therapy. Immunoassays are also used for the detection of environmental contaminants and for substance abuse testing. More recently, immunoassays have been used by non-technical persons in the home for private determinations of medical conditions such as pregnancy, ovulation and infection.

The ELISAs described herein are novel in that they detect antibodies directed against two specific surface antigens known to be of importance to either the development of infection or the modification of disease symptoms. The assays also appear to be more specific, and in the case of the 27-kDa antigen ELISA, more sensitive than a previously accepted assay which used a crude antigen preparation. The ELISA incubation and development protocol are standard. The use of the specific antigens (rather than a crude antigen) in an assay to detect antibodies in the serum of patients from a cryptosporidiosis outbreak is new.

Estimates of the number of persons infected each year will be facilitated by the sensitive and specific assays of the present invention for the detection of serum IgG antibodies against these two unique and highly antigenic C. parvum proteins.

Antibodies against the 27-kDa and 17-kDa antigens may also be used as markers for past infection in population-based studies of the risk factors associated with Cryptosporidium infection.

Uses for the ELISA methods include studying past infections of animal populations, characterizing the human antibody response to infection by C. parvum and estimating the number of persons currently infected by C. parvum.

### Additional Immunoassays

Preferably, the recombinant antigens described herein are used to detect C. parvum antibodies in a biological sample. The antibodies, immunoreactive with the antigenic epitope or proteins, are detected and quantified by any of a number of means well known to those of skill in the art. Alternatively, C. parvum proteins or peptides in the sample immunoreactive with antibodies, specific for the antigenic epitopes or proteins, are detected and quantified by the same or similar means well known to those of skill in the art. These methods include analytic biochemical methods such as spectrophotometry, radiography, electrophoresis, capillary electrophoresis, high performance liquid chromatography (HPLC), thin layer chromatography (TLC), hyperdiffusion chromatography, and the like, and various immunological methods such as fluid or gel precipitation reactions, immunodiffusion (single or double), immunoelectrophoresis, radioimmunoassays (RIAs), enzyme-linked immunosorbent assays (ELISAs), immunofluorescent assays, and the like.

One of skill in the art will appreciate that it is often desirable to reduce non-specific binding in immunoassays. Where the assay involves an antigen, antibody, or other capture agent immobilized on a solid substrate, it is desirable to minimize the amount of non-specific binding to the substrate. Means of reducing such non-specific binding are well known to those of skill in the art. Typically, this involves coating the substrate with a proteinaceous composition prior to introduction of the sample to the other immunoassay components. In particular, protein compositions such as bovine serum albumin (BSA), nonfat powdered milk, and gelatin are widely used.

Western blot analysis can also be used to detect and quantify the presence of an C. parvum peptide in the sample. The technique generally comprises separating sample products by gel electrophoresis on the basis of molecular weight, transferring the separated proteins to a suitable solid support (such as a nitrocellulose filter, a nylon filter, or derivatized nylon filter), and incubating the sample with the antibodies that specifically bind immunogenic C. parvum peptides. The anti-peptide antibodies specifically bind to a peptide fixed on the solid support. These antibodies are directly labeled or, alternatively, they may be subsequently detected using labeled antibodies (e.g., labeled sheep anti-mouse antibodies where the antibody to a peptide is a murine antibody) that specifically bind to the anti-peptide antibody.

Other assay formats include liposome immunoassays (LIAs), which use liposomes designed to bind specific molecules (e.g., antibodies) and release encapsulated reagents or markers. The released chemicals are then detected according to standard techniques (see, Monroe et al., (1986) Amer. Clin. Prod. Rev. 5:34-41).

The detection of antibodies or peptides in the sample is facilitated by labeling the proteins, antibodies specifically immunoreactive with the antigenic proteins, antibodies specific for the protein antibodies or a complex or combination of epitopes, polypeptides, antibodies or antibody fragments with a labeling agent. Detection may proceed by any known method, such as immunoblotting, western analysis, gel-mobility shift assays, fluorescent in situ hybridization analysis (FISH), tracking of radioactive or bioluminescent markers, nuclear magnetic resonance, electron paramagnetic resonance, stopped-flow spectroscopy, column chromatography, capillary electrophoresis, or other methods which track a molecule based upon an alteration in size and/or charge. The particular label or detectable group used in the assay is not a critical aspect of the immunoassay.

Some assay formats do not require the use of labeled components. For instance, agglutination assays can be used to detect the presence of target antibodies. In this case, protein-coated particles are agglutinated by samples containing the target antibodies. In this format, none of the components need be labeled and the presence of the target antibody is detected by simple visual inspection.

As mentioned above, depending upon the assay format, various components, including the protein, or protein antibody, may be bound to a solid surface. Many methods for immobilizing biomolecules to a variety of solid surfaces are known in the art.

### Generation of Antibodies

Antibodies that bind with specificity to the peptides described above are also provided. The antibodies include individual, allelic, strain, or species variants, and fragments thereof, both in their naturally occurring (full-length) forms and in recombinant forms. Additionally, antibodies are raised to these peptides in either their native configurations or in non-native configurations. Anti-idiotypic antibodies can also be generated. Many methods of making antibodies are known to persons of skill. The antibodies are useful as research tools for the isolation of additional quantities of the antigenic peptides and for studying the pathogenesis of HIV in general. The antibodies may also be useful therapeutically for passive immunization of an HIV-infected or otherwise immunocompromised patient.

The following discussion is presented as a general overview of the techniques available for the production of antibodies; however, one of skill will recognize that many variations upon the following methods are known.

A number of immunogens are used to produce antibodies specifically reactive with peptides. Recombinant or synthetic peptides of nine amino acids in length, or greater, selected from the peptides disclosed herein are the preferred peptide immunogens for the production of monoclonal or polyclonal antibodies. In one class of preferred embodiments, an immunogenic peptide conjugate is also included as an immunogen. The peptides are used either in pure, partially pure or impure form.

Recombinant peptides are expressed in eukaryotic or prokaryotic cells and purified using standard techniques. The peptide, or a synthetic version thereof, is then injected into an animal capable of producing antibodies. Either monoclonal or polyclonal antibodies can be generated for subsequent use in immunoassays to measure the presence and quantity of the peptide.

Methods of producing polyclonal antibodies are known to those of skill in the art. In brief, an immunogen, preferably a purified peptide, a peptide coupled to an appropriate carrier (e.g., GST, keyhole limpet hemanocyanin, etc.), or a peptide incorporated into an immunization vector such as a recombinant vaccinia virus is mixed with an adjuvant and animals are immunized with the mixture. The animal's immune response to the immunogen preparation is monitored by taking test bleeds and determining the titer of reactivity to the peptide of interest. When appropriately high titers of antibody to the immunogen are obtained, blood is collected from the animal and antisera are prepared. Further fractionation of the antisera to enrich for antibodies reactive to the peptide is performed where desired.

Antibodies, including binding fragments and single chain recombinant versions thereof, against fragments peptides are raised by immunizing animals, e.g., using immunogenic conjugates comprising a peptide fragment covalently attached (conjugated) to a carrier protein as described above. Typically, the immunogen of interest is a peptide of at least about 3 amino acids, more typically the peptide is 5 amino acids in length, preferably, the fragment is 10 amino acids in length and more preferably the fragment is 15 amino acids in length or greater. Often, the fragment is about 20 amino acids in length. The immunogenic conjugates are typically prepared by coupling the peptide to a carrier protein (e.g., as a fusion protein) or, alternatively, they are recombinantly expressed in an immunization vector. Antigenic determinants on peptides to which antibodies bind are typically 3 to 10 amino acids in length.

Monoclonal antibodies are prepared from cells secreting the desired antibody. These antibodies are screened for binding to normal or modified peptides, or screened for agonistic or antagonistic activity. Specific monoclonal and polyclonal antibodies will usually bind with a K_{D} of at least about 0.1 mM, more usually at least about 50 mM, and most preferably at least about 1 mM or better. Often, specific monoclonal antibodies bind with a K_{D} of 0.1 mM or better.

In some instances, it is desirable to prepare monoclonal antibodies from various mammalian hosts, such as mice, rodents, primates, humans, etc. Description of techniques for preparing such monoclonal antibodies are found in Kohler and Milstein (1975) Nature 256: 495-497. Summarized briefly, this method proceeds by injecting an animal with an immunogen, i.e., an immunogenic peptide of the present invention either alone or optionally linked to a carrier protein. The animal is then sacrificed and cells taken from its spleen, which are fused with myeloma cells. The result is a hybrid cell or “hybridoma” that is capable of reproducing in vitro. The population of hybridomas is then screened to isolate individual clones, each of which secrete a single antibody species to the immunogen. In this manner, the individual antibody species obtained are the products of immortalized and cloned single B cells from the immune animal generated in response to a specific site recognized on the immunogenic substance.

Alternative methods of immortalization include transformation with Epstein-Barr virus, oncogenes, or retroviruses, or other methods known in the art. Colonies arising from single immortalized cells are screened for production of antibodies of the desired specificity and affinity for the antigen, and yield of the monoclonal antibodies produced by such cells is enhanced by various techniques, including injection into the peritoneal cavity of a vertebrate (preferably mammalian) host. The peptides and antibodies of the present invention are used with or without modification, and include chimeric antibodies such as humanized murine antibodies. Other suitable techniques involve selection of libraries of recombinant antibodies in phage or similar vectors. See, Huse et al. (1989) Science 246: 1275-1281; and Ward, et al. (1989) Nature 341: 544-546.

Frequently, the peptides and antibodies will be labeled by joining, either covalently or noncovalently, a substance which provides for a detectable signal. A wide variety of labels and conjugation techniques are known and are reported extensively in both the scientific and patent literature. Suitable labels include radionucleotides, enzymes, substrates, cofactors, inhibitors, fluorescent moieties, chemiluminescent moieties, magnetic particles, and the like. Patents teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced. See, Cabilly, U.S. Pat. No. 4,816,567; and Queen et al., Proc. Nat'l Acad. Sci. USA 86: 10029-10033 (1989).

As mentioned above, the antibodies provided herein can be used in affinity chromatography for isolating additional amounts of the peptides identified herein. Columns are prepared, e.g., with the antibodies linked to a solid support, e.g., particles, such as agarose, Sephadex, or the like, where a cell lysate is passed through the column, washed, and treated with increasing concentrations of a mild denaturant, whereby purified peptides are released. In addition, the antibodies can be used to screen expression libraries for particular expression products, for example, HIV proteins. Usually, the antibodies in such a procedure are labeled with a moiety allowing easy detection of presence of antigen by antibody binding. Moreover, antibodies raised against the immunogenic peptides described herein can also be used to raise anti-idiotypic antibodies. Such antibodies are useful for detecting or diagnosing various pathological or resistance conditions related to the presence of the respective antigens.

### Immunoassay Kit

A kit useful for performing the immunoassay described above contains a coated particle, a binding molecule specific for the analyte to be detected, a detectable particle coated with a binding substance capable of binding to the binding molecule, and a porous membrane having a pore size that prevents passage of the coated particle and allows passage of the detectable particles. The coated particle may be either an analyte-coated particle or a second binding molecule-coated particle.

The kit may additionally contain equipment for safely obtaining the sample from the site of contamination, a vessel for containing the reagents, a timing means, and a calorimeter, reflectometer, or standard against which a color change may be measured. A simple, inexpensive calorimeter is preferred.

The reagents, including the coated particle, binding molecule and detectable particle are preferably lyophilized. Most preferably, the coated particle, binding molecule and detectable particle are provided in lyophilized form in a single container.

### Kits for the Detection of C. parvum

Kits for the detection of C. parvum in a biological sample are also provided. The kits contain the recombinant antigenic protein of the present invention. The kits may optionally contain an apparatus and one or more containers for obtaining and storing the sample prior to and during analysis and suitable buffers and other reagents to facilitate antibody-antigen binding and detection. Each component of each kit may be provided in separate containers or any combination of the components may be provided in a single container. The nucleic acid molecules encoding the antigenic protein are useful for the production thereof by recombinant means.

All patents, patent applications and publications cited herein are hereby incorporated by reference.

The invention is further illustrated by the following examples, which are not to be construed in any way as imposing limitations upon the scope thereof. On the contrary, it is to be clearly understood that resort may be had to various other embodiments, modifications, and equivalents thereof, which, after reading the description herein, may suggest themselves to those skilled in the art without departing from the spirit of the present invention.

### EXAMPLE 1

### Purification of Native 17-kDa antigen

C. parvum oocysts from the Maine isolate (Millard et al., JAMA. 272:23-30 (1994)) were collected from experimentally infected Holstein calves and purified as described by Arrowood and Sterling, J. Parasitol, 73:314-319 (1987). The 17-kDa antigen is actually a family of about 10 proteins and the five largest of these proteins at 16-17 kDa extract into Triton X-114.

A modification by Ko and Thompson, Anal. Biochem. 224:166-172 (1995) of the Triton X-114 detergent extraction protocol of Bordier, J. Biol. Chem. 256:1604-1607 (1981) was used to partially purify native 17-kDa antigen from a 24,000×g supernatant fraction of sonicated oocysts (crude antigens) in sufficient quantities for analysis and peptide sequencing (Moss et al., Am. J. Trop. Med. Hyg. 49, 393-401 (1993)).

Crude antigen at 1-3 mg/ml was mixed with an equal volume of extraction buffer to achieve final concentrations of 20 mM HEPES at pH 7.4, 150 mM NaCl, 2% Triton X-114, 1 mM phenylmethylsulfonyl fluoride (PMSF), 1 mM p-chloromercuribenzenesulfonic acid (PCMBS), and 5 mM EDTA. After a 30 minute incubation at 4° C., insoluble material was removed by centrifugation at 12,000×g for 15 minutes at 4° C. The supernatant was frozen for 24 hours at −20° C., thawed at 4° C., mixed well, and subjected to two rounds of phase partitioning at 37° C. for 10 minutes. The detergent-rich phase from the final partition was dissolved in 20 mM HEPES and 150 mM NaCl and centrifuged at 12,000×g for 15 minutes at 4° C. Partially purified antigens in the collected supernatant were precipitated with 4 volumes of acetone at −20° C. overnight. The precipitated proteins were collected by centrifugation at 12,000×g for 15 minutes at 4° C. and dried at room temperature. For use in ELISA assays, the pellet was dissolved in either a non-reducing buffer for SDS polyacrylamide gel electrophoresis (Laemmli, Nature 227:680-685 (1970)) or in a minimum volume of buffer containing 0.5% SDS and 20 mM HEPES at pH 7.4. Both solutions were heated at 95° C. for 5 minutes to insure solubilization.

Crude antigen from the Iowa isolate was used for all of the Western blot analyses of serum samples. Crude antigen from both the Iowa and Maine isolates was used for the development of the Triton X-114 extraction procedures. Triton X-114 purified antigen from the Maine isolate was used for all of the ELISA assays.

Proteins were collected from the Triton X-114 extract by acetone precipitation, then resolved under non-reducing conditions on a 10% to 22.5% SDS polyacrylamide gel using the Laemmli buffer system (Nature 227:680-685 (1970)).

A gel containing 100 μg of extracted protein (BCA protein microassay, Pierce Chemical Company, Rockford, Ill.) was stained with Coomassie brilliant blue R-250. The two most intensely stained of the five bands visible in the 17-kDa size range were excised together and submitted to the Harvard Microchemistry Facility (Cambridge, Mass.) for trypsin digestion, HPLC fractionation, and peptide sequencing. Similarly, a gel containing 37 μg of extracted protein was transferred onto a polyvinylidene difluoride (PVDF) membrane (Immobilon P, Millipore Corporation, Bedford, Mass.). The blot was stained with Coomassie blue, and the three most intensely stained proteins in the 17-kDa size range were excised together and submitted for amino-terminal peptide sequencing to the Biotechnology Core Facility Branch of the Centers for Disease Control and Prevention.

### EXAMPLE 2

### Cloning and Sequencing of the 17-kDa antigen

The partially purified 17-kDa antigen from C. parvum oocysts described in Example 1 was also used for amino-terminal peptide sequencing of all of the identified trypsin cleavage fragments shown in Table 2. The recombinant mature Cp17 has the same amino acid sequence as the native antigen. Post-translational Ser/Thr modifications are shown in bold.

Degenerate deoxyoligonucleotides pat4 (5′-CGC GGA TCC TTY GCN TTY ACN CTN GAY GG-3′) (SEQ ID NO:10) and p86r (5′-GCG GAA TTC NAC NGT RTT RTC YTT RTC NAC-3′) (SEQ ID NO:11) were designed from the peptide sequences obtained from the amino-terminus of the native protein (FAFTLDG) (SEQ ID NO:12; amino acid nos. 232-238 of SEQ ID NO:2) (forward primer) and from an internal trypsin fragment (VDKDNTV) (SEQ ID NO:13; amino acid nos. 289-295 of SEQ ID NO:2), (reverse primer), respectively.

These primers were used to PCR amplify several fragments of the Cp17 coding sequence from a C. parvum genomic library in Lambda FIX II vector (Stratagene, La Jolla, Calif.) (library provided by Norman Pienazek, CDC, Atlanta, Ga.). AmpliTaq Gold DNA polymerase was used as directed by the manufacturer (Perkin-Elmer Cetus, Foster City, Calif.) with 100 μM concentrations of both pat4 and p86r. A 192 bp fragment of the Cp17 coding sequence (Cp17 fragment) was amplified. The PCR product was digested with EcoRI and BamHI and cloned into pBluescript II SK⁺ plasmid (Stratagene), and the resulting clone was sequenced by the Emory University Sequencing Core Facility (Atlanta, Ga.). The sequence of the mature recombinant 192 bp fragment (SEQ ID NO:14; nucleic acid nos. 983-1174 of SEQ ID NO:1) is shown in FIG. 1 between the pat4 and p86r primers except for a G to A substitution at position 1079. The deduced amino acid sequence of this partial coding sequence clone (SEQ ID NO:2, shown in parenthesis and in bold in FIG. 1) was found to include all of the peptide sequence from fragments 82 and 95/99 and portions of the sequence from fragments 86 and 128/132 as expected from the placement of the primers.

In order to obtain the complete open reading frame (full-length clone), a Southern blot of EcoRI digested C. parvum genomic DNA was probed with a fluorescein-labeled non-degenerate deoxyoligonucleotide designed from the Cp17 fragment sequence insert described above (17probe2: 5′-GGT GTC TAC AGG TTG AAT GAG AAC GGA GAC TTG-3′, SEQ ID NO:16; nucleic acid nos. 1121-1153 of SEQ ID NO:1). When developed with the Southern-Light detection kit as directed by the manufacturer (Tropix, Bedford, Mass.), a single band at approximately 1.5 kb was visible. A size selected library (1-3 kb) was constructed in pBluescript SK⁺ and was screened with the fluorescein-labeled 17probe2. Clone 4.3.2 was found to contain two EcoRI fragments: a 1.33 kb fragment that included the Cp17 protein coding sequence and an unrelated 1.07 kb fragment.

The 1337 bp restriction fragment was size selected, cloned, and sequenced (SEQ ID NO:1; FIG. 1). This sequence has been assigned GenBank accession number AF114166. A 975 bp open reading frame found within this fragment encodes a putative 33.5-kDa protein that includes the mature amino terminus of the Cp17 and all of the trypsin fragments. No sequence homology was found when the recombinant Cp17 open reading frame was compared with the sequences present in the GenBank database. Two potential methionine initiation codons (Met-1 at 290 bp and Met- 131 at 660 bp) are located upstream and in-frame with the mature protein sequence. Met-1 is likely to be the initiation codon based on the following observations: the entire open reading frame could be PCR amplified with no apparent introns from a cDNA library; Met-1 is the first potential initiation codon downstream from an in-frame stop codon (TAA at 275 bp); and the bases surrounding the ATG (A at −3 and A at +4 relative to the ATG) conform with those sequences found around other eukaryotic initiation codons including those reported for C. parvum (Kozak, Nucl Acids Res. 15:8125-8148 (1987); Khramtsov et al., Res. Commun. 230:164-166 (1997)).

The sequence in FIG. 1 appears to contain both an amino-terminal 19 amino acid signal peptide (from Met-1 to the arrow in SEQ ID NO:1 of FIG. 1) for import into the endoplasmic reticulum and a hydrophobic 20 amino acid carboxy-terminal signal peptide (indicated in FIG. 1 by italics) for recognition by the GPI transamidase. The amino-terminal signal peptide with a cleavage site between Ser-19 and Ala-20 (indicated by arrow in FIG. 1) was predicted by the SignalP V1.1 program of Nielsen et al., Prot. Eng. 10, 1-6 (1997).

The bracketed region in FIG. 1 contains the two most likely ω sites for attachment of a GPI anchor to the mature protein: Asp-299 and Ser-302 (Udenfriend and Kodukula, Methods Enzymol. 250:571-582 (1995)). The mature protein from Glu-221 (circled in FIG. 1) to the putative GPI anchor addition site at Asp-299 (sequence indicated by bold letters in FIG. 1) has a predicted molecular weight of 8.5 kDa and has a predicted pI of 4.2. The difference between the observed 16-17 kDa apparent molecular weight of the native antigen and the estimated molecular weight of the recombinant protein may be attributed to a GPI anchor bound to the native Cp17 and possibly to direct glycosylation or other O-linked modification of the peptide backbone.

The mature Cp17 protein, whether recombinant or native, contains numerous serines and threonines that might serve as potential modification sites, and two observations support the concept. First, three of the four positions of ambiguity between the direct peptide sequence shown in Table 2 as SEQ ID NO:3 and the deduced protein sequence are at positions of a serine (Ser-223 in fragment No. 132; SEQ ID NO:4) and two threonines (Thr-228 and Thr-235 in fragment No. 128; SEQ ID NO:5) Second, the molecular weights (as determined by matrix-assisted laser desorption time-of-flight mass spectrometry) (MALDI-TOF MS) of the amino terminal trypsin fragments containing the ambiguous sequences (2811 daltons for fragment 128 and 2654 daltons for fragment 132), are significantly larger than the 2200 dalton mass predicted from the amino acid sequence. In contrast, the MALDI-TOF MS-determined molecular masses for fragments 86 (1901.6) and 95 (2843.0) are fully consistent with the predicted amino acid sequences of these fragments. The pI, banding pattern, and galactosamine content of the 5C3 target found in total oocyst antigen are consistent with the native GPI-anchored antigen identified (Tilley et al., Immun. 59:1002-1007 (1991); Tilley and Upton, J. Protozool. 38:48S-49S (1991)).

### EXAMPLE 3

### Expression of Recombinant Cp17

The portion of the recombinant Cp17 gene sequence that coded for the mature 17-kDa antigen from Glu-221 to Asp-299 was PCR amplified from the C. parvum genomic library as described above using deoxyoligonucleotides mat-f (5′-CGC GGA TCC GAA ACC AGT GAA GCT GCT GC -3′; SEQ ID NO:17) and mat-r (5′- GCG GAA TTC TTA ATC CTT CAA AAG AAC TGT G -3′; SEQ ID NO:18). The mature Cp17 PCR product and the Cp17 fragment PCR product (Cp17 fragment) described above were both ligated into EcoRI and BamHIIl digested pGEX 4T-2 expression vector (Pharmacia Biotech, Piscataway, N.J.) and transformed into the HB101 Escherichia coli cell line. A Schistosoma japonicum glutathione-S-transferase (GST)/Cp17 fusion protein was identified at an approximate molecular weight of 35 kDa for each PCR product. The fusion proteins were purified from isopropyl-β-D-thiogalactopyranoside induced cell cultures using glutathione Sepharose 4B, eluted from the resin with 10 mM glutathione, dialyzed to remove glutathione, and cleaved overnight at room temperature with thrombin as directed by the manufacturer (GST Bulk Purification Module, Pharmacia Biotech). Residual fusion protein and the GST cleavage product were removed from the mature Cp17 and Cp17 fragment by passage over a second glutathione Sepharose 4B column.

### EXAMPLE 4

### Immunologic Characterization of Recombinant Cp17

Various antibodies were used to compare the recombinant Cp17 antigen to the native 17-kDa antigen.

### Monoclonal Antibodies

In the first comparison, a mouse monoclonal antibody developed against the native 17-kDa antigen (C6C 1; Arrowood, Ph.D. thesis, University of Arizona, Tuscon, Ariz. (1988)) was used to probe a Western blot of the recombinant Cp17 protein.

Polyvinylidene difluoride membrane (PVDF) (Immobilon P, Millipore Corp., Bedford, Mass) strips blotted with crude oocyst antigen (600 ng/mm of gel) from a 10% to 22.5% SDS polyacrylamide gel (described above) and the mature recombinant Cp17 fragment protein (50 ng/mm of gel) were resolved on a 15% SDS polyacrylamide gel. The strips were incubated overnight at 4° C. with the eluted monoclonal antibodies, C6C1 (Arrowood, Ph.D. thesis University of Arizona, Tuscon, Ariz. (1988)), and 27-kDa antigens, C686 (Mead et al., J. Parasitol 74: 135-143 (1988)) (1:1 dilution of tissue culture supernatants in TPBS). Bound monoclonal antibody was visualized with a horseradish peroxidase-labeled goat anti-mouse polyclonal antibody.

The Western blot gel showed that monoclonal antibody C6C1 recognized both the native antigen and the mature fragment Cp17. Monoclonal antibody C6B6, which recognizes the unrelated native 27-kDa antigen, did not recognize the mature recombinant Cp17 antigen.

### Polyclonal Antibodies

To further compare the native 17-kDa antigen and the recombinant Cp17 antigen, the recombinant Cp17 fragment protein was used to affinity purify IgG antibodies from the serum of cryptosporidiosis patients.

The mature recombinant Cp17 fragment protein as isolated in Example 3 was transferred onto a PVDF membrane. The edges of the blot were stained with AuroDye Forte (Amersham Life Science, Cleveland, Ohio) and used as guides to excise the recombinant Cp17 band. The immobilized recombinant Cp17 was incubated 1 hour at room temperature with a serum from a cryptosporidiosis patient that had been diluted 1:10 in buffer containing 0.3% Tween-20, 0.85% NaCl, and 10 mM Na₂HPO₄ at pH 7.2 (TPBS). After two washes in TPBS, the bound antibodies were eluted for 15 minutes at 37° C. with an elution buffer containing 3 M MgCl₂, 25% ethylene glycol, and 75 mM HEPES/NaOH at pH 7.2 (Tsang and Wilkins, J. Immunol. Methods 138:291-299 (1991)). The antigen strip was removed, washed twice in TPBS, and cycled through the serum and elution buffer two additional times. Eluted antibodies were desalted using Sephadex G-25M size exclusion columns (PD-10, Pharmacia Biotech) pre-equilibrated in TPBS.

The recombinant Cp17 fragment protein (GenBank accession number AF097741) used in this assay (SEQ ID NO:15; nucleotides 232-295 of SEQ ID NO:2; shown in parentheses in FIG. 1) was shorter than the predicted mature recombinant Cp17 protein (SEQ ID NO:19; nucleotides 221-299 of SEQ ID NO:2; indicated in bold in FIG. 1) by 11 residues on the amino terminus and by 4-7 residues on the carboxy terminus (depending on the GPI anchor addition site).

The bound human IgG antibodies were visualized using a biotin-labeled mouse anti-human IgG monoclonal antibody (clone HP6017, Zymed Laboratories, South San Francisco, Calif.) and alkaline phosphatase-labeled streptavidin (Life Technologies, Gaithersburg, Md.) as described in Example 12.

By crude antigen Western blot, the purified polyclonal antibodies and unfractionated human serum from a different cryptosporidiosis patient recognized the same number of crude native 17-kDa antigens in the same relative ratios and at the same apparent molecular weights.

### Serum Samples

Banked serum specimens collected in 1988 were available for analysis from 74 employees at the Centers for Disease Control and Prevention (CDC) who had no history of foreign travel and no documented exposure to C. parvum (referred to as the non-outbreak serum set). Banked specimens were also available from individuals known to have been exposed to Cryptosporidium during waterborne outbreaks: 129 from the 1987 outbreak in Carrollton, Ga., (Hayes et al., N. Engl. J. Med. 320:1372-1376 (1989)) and 35 from the 1994 waterborne outbreak in Walla Walla County, Wash. (Dworkin et al., J. Infect. Dis. 174:1372-1376 (1996)). The 129 serum samples from the Georgia outbreak were divided into two sets. The Georgia “early outbreak” serum set consisted of eight samples collected from individuals 2-26 days before symptom onset and 25 samples collected from symptomatic individuals at or less than ten days after the onset of their diarrheal illnesses. Of these 33 samples, 22 were collected from individuals with laboratory-confirmed cases of cryptosporidiosis. The Georgia “late outbreak” set consisted of 76 samples from patients who met the clinical case definition for cryptosporidiosis (collected between 28-66 days after the onset of their diarrheal illness) and 20 samples from asymptomatic individuals who were exposed to the contaminated water supply (collected approximately four weeks after the outbreak) (Hayes et al., N. Engl. J. Med. 320:1372-1376 (1989)). Paired samples were available from four symptomatic individuals.

Of the 35 samples in the Washington outbreak serum set, 25 were collected from individuals who met the clinical case definition for infection with C. parvum, and ten samples were collected from exposed individuals who were asymptomatic or who had mild symptoms that did not meet the case definition (Dworkin et al., J. Infect. Dis. 174:1372-1376 (1996)). Four of the serum donors who met the clinical case definition had Cryptosporidium oocysts detected in their stool. All of the samples in the Washington outbreak set were collected approximately six weeks after the peak of the epidemic.

### Western Blot Assay

Paired sera from the two cryptosporidiosis patients were then compared by Western blot assay using both total crude antigen and recombinant mature Cp17 to determine whether antibodies to the protein component of the native 17-kDa antigen develop upon primary infection with C. parvum. The paired sera were collected from two patients from the Carrollton, Ga., waterborne cryptosporidiosis outbreak of 1987 (Hayes et al., N. Engl. J. Med. 320:1372-1376 (1989)) (1:100 dilutions TPBS). One of the two patients had oocysts detected in stool samples. Sera were collected from the stool-confirmed patient on days 2 and 68 after symptom onset. Sera were collected from the other patient on days 0 and 45 after symptom onset. These two cryptosporidiosis patients were chosen because their initial serum specimens were devoid of any Cryptosporidium-specific antibodies by Western blot assay (IgG, IgA, and IgM) and by ELISA assay (IgG).

Seroconversion for the native 17-kDa antigen as detected by crude antigen Western blot was paralleled by seroconversion for the recombinant mature Cp17 protein.

### EXAMPLE 5

### Molecular Weight of Recombinant Mature Cp17 Protein

The gel of Example 4 showed that the recombinant mature Cp17 protein migrated anomalously on gradient SDS polyacrylamide gels. The 14-16 kDa apparent molecular weight was significantly above the expected 8.5 kDa size but below the 16-17 kDa size observed for the native Triton-soluble antigens. The recombinant protein appears as multiple bands on the gradient gel.

### EXAMPLE 6

### Demonstration of Presence of GPI Membrane Anchor on Native Cp17

Various chemical and enzymatic reagents were used to demonstrate the presence of a GPI membrane anchor on the native 17-kDa antigen.

### Removal of Carbohydrates

In order to chemically remove carbohydrates from the native Cp17 antigen, crude C. parvum antigens and Triton X-114 extracted antigens were both treated for 4 hours at −20° C. with anhydrous trifluoromethanesulfonic acid (TFMS) as directed by the manufacturer (Glycofree Deglycosylation Kit, Oxford Glycosystems, Rosedale, N.Y.). The treated crude antigens were then re-extracted with Triton X-114. Aliquots of each fraction were acetone precipitated, resolved on a 10% to 22.5% SDS polyacrylamide gel, transferred onto PVDF, and either incubated with human serum from a cryptosporidiosis patient or stained with AuroDye Forte. Bound IgG antibodies were visualized as previously described.

The extracted 17-kDa antigens decreased in size to 14-15 kDa, but the overall pattern and relative intensities of the five bands were maintained. Under identical conditions, a control protein, RNase B, shifted down to the expected size for the deglycosylated product and showed no evidence of protein degradation. Deglycosylation of total crude antigen resulted in the same overall pattern in the 14-15 kDa size range. When a sample of deglycosylated total crude antigen was extracted with Triton X-114, the 17-kDa antigens partitioned to the aqueous phase rather than to the Triton X-114 phase. Such behavior is expected if the antigen was GPI-anchored and if it lacked transmembrane anchoring domains.

### Removal of Ester-linked Fatty Acids

Methanolic ammonia was used to remove ester-linked fatty acids from the native antigen (Menon, Methods Enzymol. 230:418-442 (1994)). Samples of acetone precipitated, Triton X-114 extracted antigens were incubated for 2 hours at 37° C. with either methanol or methanol-30% ammonia (1:1 by volume). Samples were dried under vacuum at room temperature, and either extracted with Triton X-114 or dissolved directly in SDS buffer for electrophoresis. Proteins were resolved on a gradient SDS polyacrylamide gel, transferred to PVDF, and blotted with either serum from a cryptosporidiosis patient or with monoclonal C6C1. Bound antibodies were visualized as described above.

Methanolic ammonia, but not methanol alone, caused the native antigen to shift up in apparent molecular weight. Methanol serves as a control for the reaction. Ammonia is the active chemical agent that removes the fatty acids. As with the deglycosylated antigens, base treated antigens were no longer extracted by Triton X-114 and multiple bands were apparent. Taken together, the observed changes in Triton X-114 solubility of the 17-kDa antigen following deglycosylation and base treatment are consistent with the presence of lyso-acyl- or diacyl-glycerol on the GPI anchor.

### Treatment with GPI-PLD

Triton X-114 extracted native Cp17 was treated with two enzymes that should remove a GPI anchor. Acetone precipitated protein was dissolved in 50 μl buffer containing 50 mM Tris (pH 7.4), 10 mM NaCl, and 0.1% sodium deoxycholate (Deeg and Davitz, Methods Enzymol. 250:630-640 (1995)). This buffer was supplemented with 2 mM CaCl₂, 5 mM EDTA or 1 mM o-phenanthroline. Following the addition of 2 μl of normal human serum containing glycosylphosphatidylinositol-specific phospholipase D (GPI-PLD), samples were incubated for 1 hour at 37° C. The proteins were precipitated with acetone, resolved by gradient SDS polyacrylamide gel electrophoresis, transferred to PVDF and visualized with monoclonal antibody C6C1 as described above.

Triton-extracted native 17-kDa antigens were completely sensitive to cleavage by the GPI-PLD found in human serum. All five of the proteins shifted down by about 1 kDa each while maintaining the same overall pattern and relative intensities of the untreated antigens. As expected for a divalent cation-dependent phospholipase (Deeg and Davitz, Methods Enzymol. 250:630-640 (1995)), cleavage with GPI-PLD could be completely inhibited by inclusion of EDTA or o-phenanthroline in the reaction buffer.

### Treatment with PI-PLC

Phosphatidylinositol-specific phospholipase C (PI-PLC) treatment of acetone precipitated native Cp17 was carried out in 50 μl buffer containing 0.1 M Tris (pH 7.4), 0.1% sodium deoxycholate, and 5 mM EDTA. Recombinant Bacillus thuringiensis PI-PLC (Oxford Glycosystems, Lot#95058 11) was added (1 pl at >250 units/ml), and the sample was incubated for 1 hour at 37° C. Presence of a serine protease was ruled out by the inclusion of 1 mM phenylmethylsulfonyl fluoride (PMSF) in one reaction.

This treatment of Triton-extracted native 17-kDa antigen with recombinant B. thuringiensis PI-PLC resulted in a shift down in apparent molecular weight of a small proportion of the antigen. The observed shift in apparent molecular weight was not sensitive to the addition of the serine protease inhibitor phenylmethylsulfonyl fluoride. Although the amount of product was quite variable between different lots of enzyme having different specific activities, its formation was inhibited by the addition of 10 mM ZnCl₂ as expected (Taguchi et al., Biochim. Biophys. Acta. 619:48-57 (1980)). The small amount of 17-kDa antigen product that was formed was not recognized by an anti-CRD antibody (from two different sources) (Zamze et al., Eur. J. Biochem. 176:527-534 (1988)), even though the anti-CRD antibody functioned as expected when Trypanosoma brucei variant surface glycoprotein was used as substrate for the recombinant PI-PLC. Cleavage with B. thuringiensis PI-PLC could not be enhanced by pretreatment of soluble or membrane bound antigen with weak base to remove ester-linked palmitate (or other fatty acids) from the inositol ring of the GPI-anchor (Mayor et al., J. Biol. Chem. 265:6174-6181 (1990); Guther et al., Anal. Biochem. 219:249-255 (1994)).

To test for divalent zinc inhibition of PI-PLC cleavage, reactions were carried out in 0.1 M Tris (pH 7.4), 0.05% Triton X-100, 1.0 mM PMSF, and either 10 mM EDTA or 10 mM ZnCl₂. Bacillus cereus PI-PLC (Beohringer Mannheim Corp., Indianapolis, Ind.) and T. brucei glycosylphosphatidyinositol phospholipase C (GPI-PLC) (Oxford Glycosystems) were used according to the manufacturer's specifications. Proteins were analyzed as with the GPI-PLD treatment. Reaction products were also probed for the presence of the anti-cross-reacting determinant (anti-CRD) by Western blot using polyclonal rabbit antisera obtained commercially from Oxford Glycosystems and with antisera kindly provided by Dr. Paul Englund (Johns Hopkins University, Baltimore, Md.). No products were visible when the antigen was treated with B. cereus PI-PLC or with T. brucei GPI-PLC. The PI-PLC results showed no/low sensitivity to cleavage.

Unusual GPI anchors have reported from other protozoa. Butikofer and Boschung, Mol. Biochem. Parasitol. 74:65-75 (1995) were unable to demonstrate anti-CRD antibody reactivity to several antigens from Herpetomonas davidi following cleavage with B. thuringiensis PI-PLC. Similarly, Das et al., J. Biol. Chem. 266:21318-21325 (1991) have reported a 49-kDa GPI anchored antigen from the surface of Giardia lamblia that was sensitive to B. cereus PI-PLC treatment, but was not recognized by the anti-CRD antibody.

The five proteins at 16-17 kDa of the 17-kDa antigen family are Triton-soluble, whereas the five 14-15 kDa forms are not. The latter forms of the antigen most likely represent proteins that lack a GPI anchor. All of the proteins probably have the same mature amino terminus since only one sequence was obtained even when multiple bands were excised from a polyacrylamide gel for sequencing. The chemically deglycosylated (TFMS-treated) Triton-soluble antigen fraction also contained five bands that migrated at the same apparent molecular weights as the five 14-15 kDa antigens. Two 17-kDa antigen bands were excised for trypsin digestion and analysis and two slightly different amino terminal fragments were detected.

### EXAMPLE 7

### In Vitro Ethanolamine Labeling of the Native Cp17

To further show the presence of a GPI anchor on the native 17-kDa antigen, the antigen was labeled with ethanolamme. In in vitro culture systems for other organisms, [³H]-ethanolamine has been shown to specifically label elongation factor 1α (EF-1α) and GPI anchored proteins (Menon et al., Methods Enzymol. 230:418-442 (1994)). Native 17-kDa antigens were reproducibly labeled to a low level using short-term in vitro cultures of C. parvum in MDCK cells.

In vitro Cryptosporidium cell culture was performed as described by Arrowood et al. in J. Euk. Microbiol 41:23S (1994) and in FEMS Microbiol. Lett. 136:245-249 (1996) in 2-well coverslip culture chambers (Nunc, Naperville, Ill.). Briefly, MDCK cell monolayers grown in serum free medium (Ultraculture, BioWhittaker, Walkersville, Md.) were infected with 2×10⁶ excysted oocysts per chamber (Maine isolate) for 3 hours at 37° C. in a 5% CO₂ environment (Arrowood et al., J. Euk. Microbiol. 41:23S (1994)). After washing to remove oocysts and unattached sporozoites, each cell culture was incubated with 2 ml of fresh medium for an additional 4 hours at 37° C. in a 5% CO₂ environment. The Ultraculture medium was then replaced with 1 ml of fresh RPMI medium supplemented with 7.5 mM HEPES at pH 7.4, 25 mM glucose, 100 units/ml penicillin G, 100 μg/ml streptomycin sulfate, and 200 μCi/ml [¹³H]ethan-1-ol-2-amine hydrochloride (18 Ci/mmol, Amersham Pharmacia Biotech, Piscatawag, N.J.). Cultures were maintained for 48 hours at 37° C. in a 5% CO₂ environment. Cultures were harvested, centrifuged for 5 minutes at 2000×g at room temperature, and washed once with fresh Ultraculture medium at 4° C.

Each cell pellet from the labeling experiment was resuspended in 200 μl of buffer containing 20 mM HEPES at pH 7.4, 150 mM NaCl, 2% Triton X-114, 1 mM phenylmethylsulfonyl fluoride (PMSF), 1 mM p-chloromercurybenzenesulfonic acid, and 5 mM EDTA. The cell suspension was frozen in a dry ice/ethanol bath and thawed at 37° C. for two cycles then extracted with Triton X-114 as described in Example 1. The Triton soluble proteins were acetone precipitated and redissolved in 25 μl of SDS sample buffer under nonreducing conditions. Proteins were resolved on a 10% to 22.5% SDS polyacrylamide gel, transferred to PVDF membrane, and subjected to fluorography using EN³HANCE spray (NEN Life Science Products, Boston, Mass.). After fluorography, the fluor was removed by rinsing with methanol and the membrane was blotted with monoclonal antibodies that recognize the 17-kDa antigen (C6C1) and the 27-kDa antigen (C6B6). Western blots were developed as described earlier.

Triton X-114 extraction was used to separate the 17-kDa antigen from any EF-1α that might also have been labeled during the incubation, and acetone precipitation was used to separate the antigen from most of the labeled lipids. Two bands in the 17-kDa size range were visible only in MDCK cells that had been infected with Cryptosporidium, and these bands were exactly coincident with the largest of the two of the 17-kDa antigen protein bands detected by monoclonal antibody C6C1.

In summary, the identification of the recombinant Cp17 as functionally equivalent to the immunodominant 17-kDa antigen was further confirmed by the demonstration of a GPI anchor on the native antigen. Of all the cloned C. parvum antigens in this size range, only the recombinant Cp17 has a putative GPI targeting sequence. The ability to label the native 17-kDa antigen with ethanolamine, the observed shift in apparent molecular weight following serum GPI-PLD treatment, and the changes in Triton X-114 solubility upon deglycosylation and treatment with weak base are all consistent with a GPI anchor having a lyso-acyl or diacyl-glycerol.

### EXAMPLE 8

### Western Blot of Serum Samples from Symptomatic and Asymptomatic Carrollton, Ga. Residents for Testing Sensitivity and Specificity

The crude antigen supernatant prepared from sonicated oocysts was resolved on a 10% to 22.5% SDS polyacrylamide gel under nonreducing conditions (600 ng/mm of gel width). Following electrotransfer to Immobilon P, 2-mm strips of membrane were incubated overnight at 4° C. with serum from individual cryptosporidiosis patients (described in Example 4) at a dilution of 1:100 in 0.3% Tween-20/PBS. The blots were developed with a biotinylated anti-human IgG monoclonal antibody and alkaline phosphatase-labeled streptavidin. Total unfractionated proteins (Ttl), proteins found in the detergent-depleted aqueous phase (AQ), and proteins extracted into the Triton X-114 detergent-rich phase (TX) were resolved. Antigens were recognized by serum collected from cryptosporidiosis patients 28-66 days after symptom onset (late outbreak). Antigens were also recognized by sera collected from patients less than 10 days after symptom onset (early outbreak).

It was seen that the 27-kDa antigen family contains approximately 5 proteins in the 23- to 27-kDa size range, and the 17-kDa antigen family contains approximately 10 proteins in the 15- to 17-kDa size range. Western blots showed IgG antibodies against the 27- and 17-kDa antigen families were consistently detected in late outbreak serum samples collected from the Georgia case-patients 28-66 days after diarrheal onset. No other antigens were as consistently recognized by this battery of serum samples. Furthermore, antibodies against these antigens were detected more frequently in the late outbreak sera than in early outbreak sera collected from Georgia case-patients. These observations suggested that antibodies against the two antigens might serve as useful markers for past infection with the parasite.

The Ttl, AQ and TX proteins were blotted onto Immobilon P and incubated overnight at 4° C. with either: a 1:100 dilution of human serum from a cryptosporidiosis patient in 0.3% Tween-20 PBS (Human serum); a 1:1 dilution of tissue culture supernatant in 0.3% Tween-20 PBS containing a monoclonal antibody (C6C1) against the 17-kDa antigen (anti-17-kDa mAb); a 1:1 dilution of tissue culture supernatant in 0.3% Tween-20 PBS containing a monoclonal antibody (C6B6) against the 27-kDa antigen (anti-27-kDa mAb); or AuroDye-forte to stain all proteins (AuroDye). Blots were developed with either a biotinylated anti-human IgG monoclonal antibody and streptavidin-labeled alkaline phosphatase (human serum) or a horseradish peroxidase-labeled goat anti-mouse polyclonal antibody (anti-17-kDa mAb and anti-27-kDa mAb).

The 17- and 27-kDa antigens recognized by serum samples from an infected patient were partitioned unequally between the two phases following detergent extraction. Of the family of ten 17-kDa antigens recognized by the C6C1 monoclonal antibody, the five largest proteins were completely extracted into the detergent phase, leaving five proteins in the 15-kDa size range in the aqueous phase. The proteins were recognized by the monoclonal antibody even after total chemical deglycosylation with anhydrous trifluoromethanesulfonic acid (TFMS). Similarly, of the family of five 27-kDa antigens recognized by the C6B6 monoclonal antibody, the three proteins with the highest apparent molecular weight were extracted into the detergent phase, leaving two lower molecular weight proteins in the aqueous phase. These proteins were also recognized by the monoclonal antibody following total chemical deglycosylation.

Surprisingly, the majority of the oocyst antigens that were recognized by the antibodies in human post-infection serum remained in the aqueous phase following extraction. Many of these antigens are clearly glycosylated, since cleavage of the carbohydrate epitopes with periodate significantly reduced the Western blot reactivity in the high molecular weight range without affecting serum antibody binding to the 17-and 27-kDa antigens. An AuroDye stained blot of the Triton X-114 extraction suggested that a total of about 10-20 proteins were partitioned into the detergent phase. This represents about 3% of the total protein present in the initial oocyst sonicate (Micro BCA protein assay, Pierce Chemical Company, Rockford, Ill.).

### EXAMPLE 9

### Eliciting an Antibody Response Against Recombinant Cp17

One New Zealand white rabbit was immunized with purified recombinant Cp17 using the following schedule:

| | |
|---|---|
| day 0 | 60 micrograms in Freund's complete adjuvant |
| day 14 | 30 micrograms in Freund's incomplete adjuvant |
| day 35 | 30 micrograms in Freund's incomplete adjuvant |
| day 49 | 30 micrograms without adjuvant |

Serum was collected at the indicated time point and was analyzed for the presence of IgG antibodies that recognize the native 17-kDa C. parvum antigen using both the Western blot and Triton antigen (partially purified native 17-kDa antigen) ELISA.

| | | ELISA response |
|---|---|---|
| Day | Blot response | (Optical density at 405 nm) |
| 0 | Negative | 0.005 |
| 14 | Weak positive | 0.028 |
| 35 | Strong positive | 0.530 |
| 49 | Not assayed | 0.732 |
| 67 | Not assayed | 0.847 |
| 128 | Not assayed | 0.926 |

### EXAMPLE 10

### Recombinant 27-kDa Protein Expression

The 11.2 kDa coding sequence identified by Perryran et al. (Perryman et al., Mol. Biochem. Parasitol. 80:137-147 (1996)) as the 27-kDa antigen was cloned into the pGEX expression vector in-frame with GST.

The following two deoxyoligonucleotides were designed for the directional cloning of the C. parvum 27-kDa antigen (Perryman et al., Mol. Biochem. Parasitol. 80:137-147 (1996); GenBank accession number U34390) into the BamHI and EcoRI restriction enzyme sites of the pGEX 4T-2 expression vector (Amersham Pharmacia Biotech, Piscatawag, N.J.): Cp23 5′-primer (5′-CGC GGA TCC ATG GGT TGT TCA TCA TCA AAG-3′; SEQ ID NO:20) and Cp23 3′-primer (5′-GCG GAA TTC ATT AGG CAT CAG CTG GCT TG-3′; SEQ ID NO:21).

The 27-kDa antigen coding sequence was amplified from 260 ng of genomic DNA by using 100 μM concentrations of Cp23-5′ and Cp23-3′ and AmpliTaq DNA polymerase as directed by the manufacturer (Perkin-Elmer Cetus, Norwalk, Conn.). The following amplification protocol was used: 30 cycles of 94° C. for 1 minute, 55° C. for 2 minutes, and 72° C. for 3 minutes followed by 1 cycle of 72° C. for 15 minutes.

Plasmid containing insert was transformed into Escherichia coli strain HB101 cells (Life Technologies, Frederick, Md.). The sequence of the resulting clone was confirmed by automated DNA sequencing. A recombinant C. parvum 27-kDa antigen/Schistosoma japonicum glutathione-S-transferase (GST) fusion protein was purified from isopropyl-β-D-thiogalactopyranoside (IPTG) induced cell cultures using glutathione Sepharose 4B as directed by the manufacturer (GST Bulk Purification Module, Amersham Pharmacia Biotech, Piscatawag, N.J.).

The C. parvum protein with an additional GlySer dipeptide at the amino terminus was released by overnight cleavage with thrombin at room temperature and then separated from uncleaved fusion protein and the GST cleavage product by passage over glutathione Sepharose 4B resin. Protein purity was monitored by both SDS polyacrylamide gel electrophoresis and Western blotting with a monoclonal antibody against the native 27-kDa antigen (C6B6; Mead et al., J. Parasitol. 74:135-143 (1988)) and with serum samples from infected humans.

Lane components were as follows: purified GST (lane 1), E. coli cells containing the plasmid construct before IPTG induction (lane 2), the same cells 4 hours after IPTG induction (lane 3), purified fusion protein (lane 4), purified fusion protein after partial thrombin cleavage (lane 5), 2 μg of recombinant 27-kDa antigen after removal of uncleaved fusion protein and GST (lane 6). Lane 6 was digitally enhanced to improve the visibility of the weakly staining recombinant 27-kDa protein band.

Induction with IPTG resulted in the appearance of a fusion protein band at an apparent molecular weight of 43 kDa (lane 2 and 3). In contrast to the native antigen, the fusion protein was not membrane associated in the bacterial lysate nor could it be extracted into Triton X-114. The fusion protein bound to the glutathione Sepharose 4B column and could be eluted with only a few minor contaminants in the 30- to 32-kDa size range (lane 4). Cleavage of the glutathione Sepharose 4B-bound fusion protein with thrombin (lane 5) resulted in a preparation that contained a single, weakly staining protein band at an apparent molecular weight of 27 kDa (lane 6). The approximate yield of this purified protein was 0.2 mg (Micro BCA assay, Pierce Chemical Company, Rockford, Ill.) per liter of E. coli culture.

The 27-kDa apparent molecular weight of the purified, thrombin-cleaved recombinant antigen was unexpected given the length of the coding sequence that was cloned into the expression vector. A monoclonal antibody (C6B6) raised against the native 27-kDa antigen was used in a Western blot of the proteins resolved to confirm that the protein band at 27 kDa was indeed the recombinant 27-kDa antigen. The fusion protein, like the purified antigen, reacted with the monoclonal antibody, but no reactivity was noted in the lane loaded with GST alone. The weak staining with Coomassie blue and the aberrant migration may reflect the high alanine, proline, and acidic residue content of the cloned protein sequence (25% ala, 19% pro, and 20% asp plus glu). As with the fusion protein, the purified recombinant antigen could not be extracted into Triton X-114 detergent.

### EXAMPLE 11

### Western Blot Assay for the Presence of Anti-C. parvum Antibodies

Serum sets that were available for use in the ELISA sensitivity and specificity determinations were assayed for the presence of anti-C. parvum antibodies using a modification of the Western blot assay of Moss et al. (Moss et al., Am. J. Trop. Med. Hyg. 58:110-118 (1998)).

Crude oocyst proteins from the Iowa isolate of C. parvum were resolved on 10%-22.5% SDS polyacrylamide gels using the buffer system of Laemmli (Laemmli, Nature 227:680-685 (1970)). The proteins were electrotransferred onto PVDF membrane (Immobilon P, Millipore Corp., Bedford, Mass.) and cut into 2-mm strips. Each strip was incubated overnight at 4° C. with a 1:100 dilution of serum in phosphate-buffered saline (0.85% NaCl and 10 mM Na₂HPO₄ at pH 7.2) (PBS) containing 0.3% Tween-20 detergent. Bound antibodies were detected with a biotin-labeled mouse monoclonal antibody against human IgG (clone HP6017, Zymed Laboratories, South San Francisco, Calif.) and alkaline phosphatase-labeled streptavidin (Life Technologies, Gaithersburg, Md.). Nitro Blue tetrazolium and 5-bromo-4-chloro-3- indolyl phosphate were used to visualize the bound antibodies. Presence or absence of antibodies against the 17- and 27-kDa antigens was determined visually by three independent readers. Eight ambiguous samples (3.3% of the total) were reassayed and four of these were assayed a third time until a consensus was reached by at least two of the readers.

Western blots that used a mouse monoclonal antibody for visualization of bound antibodies to the 27-kDa antigen (C6B6; Mead et al., J. Parasitol. 74:135-143 (1988)) or to the 17-kDa antigen (C6C1; Arrowood, PhD. thesis. University of Arizona, Tuscon, Ariz. (1988)) were developed with a horseradish peroxidase-labeled goat anti-mouse antibody in 0.3% Tween-20 PBS. After incubation for 1 hour at room temperature, antibodies were visualized with diaminobenzidine substrate and H₂O₂.

The Western blots described in Example 10 are representative of the early and late outbreak serum sets available from Carrollton, Ga. Of the early outbreak serum samples, 33% were positive by immunoblot for IgG antibodies to the 17-kDa antigen, and 52% were positive for antibodies to the 27-kDa antigen (Table 3). In contrast, 95% and 99% of the late outbreak serum samples were positive for antibodies against the 17-kDa and 27-kDa antigens, respectively (Table 3). No significant differences were observed between the Western blots of the serum samples from symptomatic individuals and those from asymptomatic individuals in the Georgia late outbreak set. The Washington outbreak serum set had frequencies of blot positives similar to those observed for the Georgia late outbreak set: 80% and 97% were positive for antibodies against the 17- and 27-kDa antigens, respectively (Table 3). As with the Georgia late outbreak set, no significant differences were observed between the Western blots of the serum from symptomatic and asymptomatic individuals in the Washington set. The non-outbreak serum set had an intermediate frequency of positives for antibodies against the 17-kDa antigen (62%) and a high frequency of positives for antibodies against the 27-kDa antigen (92%).

^{a-f} Frequencies indicated by the same footnotes were found to have significant differences using the Freeman-Tukey test. P values were (a) 0.0001, (b) 0.0001, (c) 0.0018, (d) 0.0002, (e) 0.0001, and (f) 0.0

### EXAMPLE 12

### Enzyme-linked Immunosorbent Assay

The serum sets were assayed by ELISA using both the Triton X-114 extracted 17-kDa antigens and the recombinant 27-kDA antigen.

Antigens diluted in 0.1 M NaHCO₃ buffer (pH 9.6) were used to sensitize 96-well plates overnight at 4° C. (Immulon 2 flat-bottom microtiter immunoassay plates, Dynatech Industries, Inc., McLean, Va.). Each well contained 50 μl of either the recombinant 27-kDa antigen (0.2 μg/ml) or the Triton X-114 extracted antigens (0.14-0.28 μg/ml) (BCA protein microassay, Pierce Biotech. Company, Rockford, Ill.). The plates were washed in 0.05% Tween-20 PBS and blocked with 0.3% Tween-20 PBS for 1 hour at 4° C. After a series of three washes (subsequent washes were all with 0.05% Tween-20 PBS), 50 μl aliquots of serum diluted 1:50 with wash buffer were added to each well. All serum samples were tested in duplicate. A two-fold serial dilution (1:50 to 1:6400) of a strong positive control was used to generate a standard curve on each individual plate. One buffer blank and a battery of seven serum samples known by Western blot assay to be negative for C. parvum antibodies were also included on each plate. Plates were incubated 2 hours at room temperature, then washed four times with wash buffer. A biotinylated mouse monoclonal antibody against human IgG (clone HP6017, Zymed Laboratories, South San Francisco, Calif., 50 μl of a 1:1000 dilution in wash buffer) was added to each well and incubated for 1 hour at room temperature. Following four washes, the wells were filled with alkaline phosphatase-labeled streptavidin (Life Technologies, Gaithersburg, Md., 50 μl of a 1:500 dilution in wash buffer) and incubated an additional hour at room temperature. After four washes (the final wash for 10 minutes at room temperature), p-nitrophenylphosphate substrate was added in 3 mM MgCl₂ and 10% diethanolamine at pH 10, and the color was allowed to develop until the 1:50 positive control wells had reached an absorbance of about 1.5 at 405 nm. Absorbances were measured using a Molecular Devices UVmax kinetic microplate reader. Antibody levels of the unknown samples were assigned a unit value based on the 8-point positive control standard curve with a 4 parameter curve fit. The 1:50 dilution of the positive control was arbitrarily assigned a value of 6400 units. Unknown samples with absorbance values above the standard curve were diluted further and reassayed. Arbitrary unit values were expressed per microliter of serum.

For the crude oocyst antigen ELISA protocol, a modification of the protocol of Ungar et al. (Ungar et al., J. Infect. Dis. 153:570-578 (1986)) was used. Each well contained 50 μl of crude oocyst antigen in bicarbonate buffer at a protein concentration of 2.0 μg/ml. Test serum samples were diluted 1:50 in 0.3% Tween-20 PBS, and the plates were developed as described above with a biotinylated monoclonal anti-IgG antibody and alkaline phosphatase-labeled streptavidin. Quantitation of the test serum samples was as described above and was based on the same positive control serum dilution.

### Statistical Analysis

ELISA absorbance values for the various sample sets were converted into unit values as described and geometric means were calculated. Geometric means were then compared by using a multiple comparison t-test. Blot positives between groups were compared with the Freeman-Tukey multiple comparisons test.

### ELISA Analysis of Serum Sets

Mean values, medians, and ranges (in arbitrary units) for the two assays are given in Table 4.

The Georgia early outbreak set had the lowest mean reactivity for both the Triton antigen (11.5 units) and the recombinant 27-kDa antigen (102.5 units). The mean ELISA values were significantly lower than those determined for the Georgia non-outbreak set (P=0.0001 and 0.0012, respectively) and the Georgia late outbreak and Washington outbreak sets (P=0.0001 for all comparisons). Mean Triton antigen and recombinant 27-kDa antigen ELISA values for the non-outbreak set were seven-fold and four-fold higher than the Georgia early outbreak set, respectively, but were still significantly lower than those determined for the Georgia late outbreak (P=0.0001 for both assays) and Washington outbreak sets (P=0.0104 and 0.0226, respectively).

In contrast to the qualitative assessment of the 27-kDa group in the Western blot assay, the recombinant 27-kDa antigen ELISA was able to demonstrate a significant difference (P=0.0243) between symptomatic and asymptomatic members of the Georgia late outbreak set. Symptomatic individuals had a mean value of 2015.7 arbitrary units (median=2060, R=87-34,456) compared to 492.6 units for the asymptomatic individuals (median=940, R=0-4599). Similar trends were noted for the Triton antigen ELISA (mean values of 454.0 and 203.2 for symptomatic and asymptomatic subsets, respectively) and for the symptomatic and asymptomatic subsets from the Washington outbreak (recombinant 27-kDa ELISA mean values of 1854.7 and 565.1; Triton antigen ELISA values of 439.2 and 147.0, respectively), but the differences did not approach statistical significance. Neither ELISA assay detected a significant difference between the Georgia late outbreak and Washington outbreak sets.

### Sensitivity and Specificity of ELISAs

Based on an overall comparison of the ELISA responses and the Western blot results (which served as the reference standard), positive threshold unit values were chosen for both ELISAs so as to maximize the sensitivity and specificity of the ELISAs relative to the Western blot. These values were generally greater than the cutoff values that would have been assigned based on a mean plus three standard deviations of the ELISA responses of the seven Western blot-negative controls that were included on each ELISA plate. Using a positive cutoff of 206 units, the recombinant 27-kDa antigen ELISA was able to predict 90% of the samples that were positive by blot for antibodies against the 27-kDa antigen and 92% of the samples that were negative by blot. Using a positive cutoff of 76 units for the Triton-purified antigen ELISA, this assay was able to correctly identify 90% of those samples that were positive by blot for antibodies against the 17-kDa antigen and 94% of the samples that were negative by blot.

The Triton antigen ELISA response did not correlate well with the 27-kDa antigen blot response even though the 27-kDa antigen was present in the partially purified preparation used to sensitize the plates. Of 39 samples that were positive by blot for antibodies against the 27-kDa antigen but negative by blot for antibodies against the 17-kDa antigen, only 3 (8%) were defined as positive using the Triton-purified antigen ELISA. Antibodies against the 27-kDa antigen were certainly present in most of these samples since 30 (77%) were correctly identified as positive when the recombinant 27-kDa antigen ELISA was used. Thus, the 17-kDa antigen appears to be responsible for most of the response seen in the ELISA using the partially purified Triton fraction.

Others (Moss et al., Am. J. Trop. Med Hyg. 58:110-118(1998), Moss et al, J. Infect. Dis. 178:827-833 (1998)) have reported that the blot responses to the 27- and 17-kDa antigens are not well correlated with the ELISA response when a crude oocyst antigen preparation is used to sensitize the plates. In our hands, a modified version of the crude antigen ELISA (at a 117-unit positive cutoff value chosen to optimize sensitivity and specificity relative to the Western blot) had a 91% sensitivity and a 60% specificity for detection of antibodies against the 17-kDa antigen. At the same unit value cutoff, the crude antigen assay accurately predicted 83% of the samples that were positive by blot for antibodies against the 27-kDa antigen and 69% of those that were negative. However, of 23 samples that were negative by blot for antibodies against both the 17- and 27-kDa antigens, 5 (22%) appeared to be positive by the crude antigen ELISA. Only one of these same samples was misclassified by the Triton antigen ELISA and only two were misclassified by the recombinant 27-kDa antigen ELISA. Of 42 samples that were positive by blot for only one antigen, the crude antigen ELISA detected 21 (50%) while the recombinant 27-kDa antigen and Triton-purified antigen ELISAs together correctly identified 30 (71%).

### ELISAs on Paired Sera

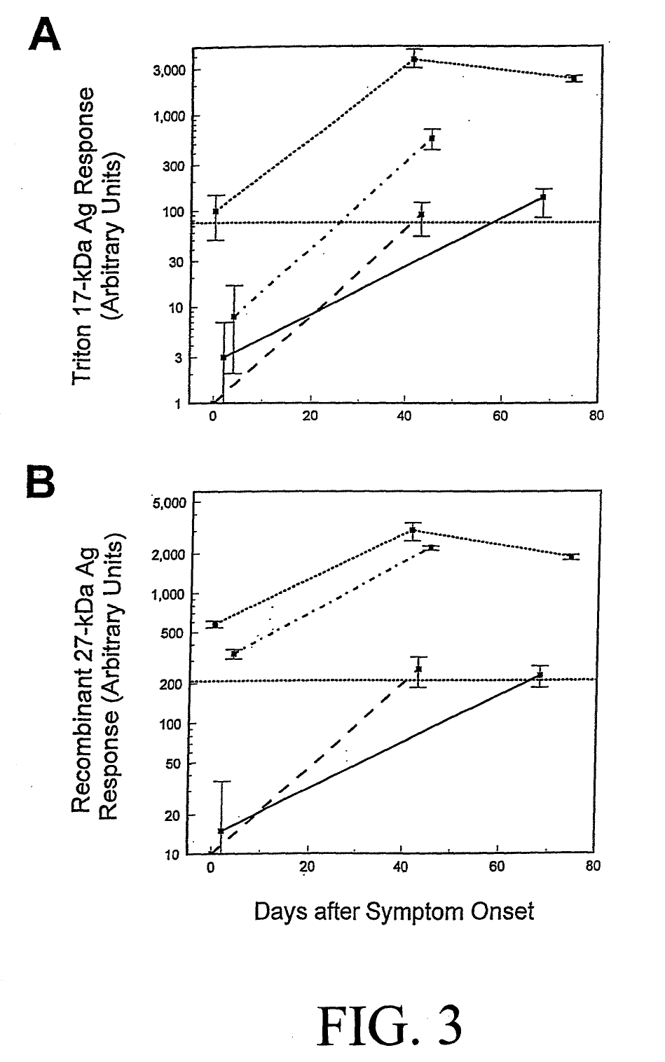

Both ELISAs were used to monitor changes in antibody levels in paired serum samples from four symptomatic individuals (two of whom had laboratory-confirmed cryptosporidiosis) from the Carrollton, Ga, outbreak. In FIG. 3 it is shown that the initial serum samples were collected in the early outbreak period (0-4 days after symptom onset), and the follow-up samples were collected in the late outbreak period (41-68 days after symptom onset). A third sample was available from one individual (74 days after onset). Two of these individuals who were initially negative for IgG antibodies by both ELISAs developed a positive response by the second time point. The seroconversion detected by ELISA for these two individuals was also evident by immunoblot. IgG antibodies against the 27- and 17-kDa antigens as well as IgA antibodies against the 17-kDa antigen and IgM antibodies against the 27-kDa antigen were present in the late outbreak serum specimens but absent from the early outbreak samples. One individual was ELISA positive by both assays at all time points and had peak levels of antibody approximately 40 days after onset. The remaining individual, who was initially negative for antibody by Triton antigen ELISA but positive by the recombinant 27-kDa antigen ELISA, was positive by both assays at the later time point. All of the individuals experienced an increase in antibody levels by both assays after the initial sample was collected. The Triton antigen ELISA and the recombinant 27-kDa antigen ELISA were able to track changes in antibody levels in single individuals and to correctly identify those individuals who had anti-17-kDa and anti-27-kDa antibodies by Western blot in each instance.

### EXAMPLE 13

### Assay Comparisons

Comparison of the data of the Triton ELISA, the total antigen ELISA and the recombinant mature Cp17 ELISA is shown in Table 5.

This comparison shows that serum IgG antibodies to the Khramtsov antigen were not detected in the majority of human cryptosporidiosis patients while antibodies against both the purified native 17-kDa antigen (Triton Ag) and the recombinant Cp17 antigen (rCp17) were demonstrated and were correlated with the presence of an antibody response by Western blot (Blot status). The results also demonstrate that the purified native 17-kDa antigen and the recombinant 17-kDa antigen are more specific compared to the Western blot response than is the crude antigen ELISA (Total Ag). Samples 14, 16, 20, and 22 appear positive by total antigen ELISA, but are negative by Western blot and by the 17-kDa antigen ELISAs.

TABLE 1

| | | Jenkins et al. | | |
|---|---|---|---|---|
| | | (‘93), | | |
| | | Khramtsov, | | |
| | | and U.S. Pat. | | |
| | | No. | | Jenkins and |
| | Recombinant | 5,591,434 | Mead | Fayer (‘95) |
| | Cp17 | antigen | antigen | antigen |
| Binds to anti- | Y | N | N | Not Assayed |
| bodies found in | | | | |
| sera of C. | | | | |
| parvum patients | | | | |
| Binds to known | Y | N | Y | Not Assayed |
| Mab to native | | | | |
| Cp17, C6C1 | | | | |
| Putative GPI | Y | N | N | N |
| anchor addition | | | | |
| sequences | | | | |
| Endoplasmic | Y | N | N | N |
| reticulum | | | | |
| targeting | | | | |
| sequence | | | | |
| Hydrophobic | N | N | N | N |
| characteristics | | | | |

TABLE 2
Peptide sequences obtained from purified native Cp17
Amino-terminal peptide sequence of mature native Cp17¹

ETDGAAAQVDLFAFQLD (SEQ ID no:3,; amino acid nos. 220-237 of
SEQ ID NO:2)

| Fragment | |
|---|---|
| Number | Peptide sequences from trypsin digested native Cp17¹ |
| 132 | ETDEAAATVDLFAFTLDGG (SEQ ID. NO:4; amino acid |
| | nos. 220-239 of SEQ ID NO:2) |
| 125 | ETXEAAA(G/S)VDLFAF(G/S)LDGGKR (SEQ ID NO:5; |
| | amino acid nos. 220-241 of SEQ ID NO:2) |
| 82 | IEVAVPNVED² (SEQ ID NO:6; amino acid nos. 242-251 of |
| | SEQ ID NO:2) |
| 95 | DKYSLVADDKPFYTGANSGTTNGVYR (SEQ ID NO:7; |
| | amino acid nos. 256-281 of SEQ ID NO:2) |
| 99 | YSLVADDKPFYTGANSGTTNGVYR (SEQ ID NO:8; |
| | amino acid nos. 258-281 of SEQ ID NO:2) |
| 86 | LNENGDLVDKDNTVLLK (SEQ ID NO:9; amino acid nos. |
| | 282-298 of SEQ ID NO:2) |

¹Amino acids indicated in bold differ from the deduced amino acid sequence shown in FIG. 1.
²Sequencing terminated after 10 cycles.

TABLE 3
Western blot characterization of serum samples

| Samples | Number of blot positives (%) | | |
|---|---|---|---|
| Serum Set | available | 17 kDa | 27 kDa |
| Georgia | 74 | 46 (62)^{a} | 68 (92)^{d} |
| Non-outbreak | | | |
| Georgia | 33 | 11 (33)^{b,c} | 17 (52)^{d,e,f} |
| Early outbreak | | | |
| Georgia | 96 | 91 (95)^{a,b} | 95 (99)^{e} |
| Late outbreak | | | |
| (total) | | | |
| Symptomatic | 76 | 73 (96) | 76 (100) |
| Asymptomatic | 20 | 18 (90) | 19 (95) |
| Washington | 35 | 28 (80)^{c} | 34 (97)^{f} |
| Late outbreak | | | |
| (total) | | | |
| Symptomatic | 25 | 22 (88) | 25 (100) |
| Asymptomatic | 10 | 6 (60) | 9 (90) |

We claim:

1. A recombinant antigenic protein of C. parvum, comprising:
glycosylphosphatidylinositol anchor site characteristics; and
characteristics of an endoplasmic reticulum directed protein
wherein the protein is immunogenic with antibodies that bind to a C. parvum antigen having an approximate molecular weight of 17-kDa.

2. The recombinant antigenic protein of claim 1 which when administered to a human or animal induces in the human or animal an immune response conferring protection against infection with C. parvum.

3. The recombinant antigenic protein of claim 2 encoded by the amino acid sequence of SEQ ID NO:2, or a mutant, variant, fragment, or synthetic protein thereof.

4. The recombinant antigenic protein of claim 3 encoded by a nucleotide sequence that expresses this protein, mutant, variant, fragment, or synthetic protein thereof.

5. The recombinant antigenic protein of claim 1 having the nucleotide sequence of SEQ ID NO:1.

6. The recombinant antigenic protein of claim 5 comprising a full-length coding sequence defined by nucleotides 290-1264 of SEQ ID NO:1.

7. The recombinant antigenic protein of claim 3 combined with an appropriate pharmaceutical carrier.

8. An antibody to the antigenic protein of claim 5, wherein the antibody is a monoclonal antibody, polyclonal antibody or chimeric construct.

9. A vaccine for immunization of a human or animal host comprising an administration to the human or animal an immunogenic amount of a C. parvum peptide or antigen described as in claim 1, or DNA or RNA encoding said peptide or antigen, capable of evoking production of anti-C. parvum antibodies.

10. The vaccine of claim 9, wherein the peptide has the amino acid sequence of SEQ ID NO:15.

11. The vaccine of claim 10 wherein the administration of the peptide, antigen, DNA or RNA evokes in the human or animal an active immunity against C. parvum infection.

12. The vaccine of claim 11 additionally containing an appropriate pharmaceutically acceptable adjuvant.

13. A method of active prophylaxis of C. parvum infection comprising administering to a subject in need of such prophylaxis an amount of the compositions of claim 1 capable of binding to an anti-C. parvum antibody, said amount of the antigen sufficient to elicit production of anti-C. parvum antibodies.

14. The method of active prophylaxis of claim 13, wherein the antigen has the amino acid sequence of SEQ ID NO:15.

15. A method for passive prophylaxis of C. parvum infection comprising administering to a subject in need of such prophylaxis an amount of an anti-C. parvum antibody that sufficiently binds the composition of claim 1 to ameliorate the infection.

16. A method for detecting C. parvum antibodies in a biological sample by combining a recombinant antigen of claim 1 with a sample and detection of an antigen-antibody complex.

17. A method for partial purification of 27-kDa and 17-kDa surface antigen proteins from C. parvum oocysts comprising the steps:
solubilizing the oocysts by protein content;
extracting the proteins with an extraction buffer comprising 20 mM HEPES, PMSF and EDTA;
separating the proteins;
resuspending the proteins in a resuspension buffer comprising 20 mM HEPES;
removing detergent by cold precipitation; and
collecting the proteins.

18. A kit for diagnosing and monitoring C. parvum infection, comprising:
recombinant 17-kDa surface antigen of claim 1 coded by the nucleic acid sequence of SEQ ID NO:1;
optionally containing an apparatus and one or more containers for obtaining and storing a sample prior to and during analysis; and
buffers and other reagents for facilitating antibody-antigen binding and detection
wherein each component of each kit may be provided in separate containers or any combination of the components may be provided in a single container.

19. A mature antigenic protein defined by SEQ ID NO:15.

20. A method of using the mature antigenic protein of claim 19 comprising replacing an extracted native antigen with the mature antigenic protein of claim 19 in an assay.


SEQUENCE LISTING (22 sequences)

SEQ ID NO: 1 - DNA, 1337 bp, Artificial Sequence
  Description of Artificial Sequence/note = synthetic construct
gaattcaatacaaagaataggactcaatataaagtcaaccttgaaattaaattaatataaatttttaagagtagactcgtacgtatgaaatgcttatcgtcttcacatgcatgcaaaaatacgtggactgggtgtatccacataaaaaagcaattaaccacattttacccacacatctgtagcgtcgtcaagtaaaaattgataacaaatttttatacattcggctcgacccttctataggtgataattagtcagtctttaataagtaggcaactaaggacaaaggaagatgagattgtcgctcattatcgtattactctccgttatagtctccgctgtattctcagccccagccgttccactcagaggaactttaaaggatgttcctgttgagggctcatcatcgtcatcgtcatcatcatcatcatcatcatcatcatcatcatcaacatcaaccgtcgcaccagcaaataaggcaagaactggagaagacgcagaaggcagtcaagattctagtggtactgaagcttctggtagccagggttctgaagaggaaggtagtgaagacgatggccaaactagtgctgcttcccaacccactactccagctcaaagtgaaggcgcaactaccgaaaccatagaagctactccaaaagaagaatgcggcacttcatttgtaatgtggttcggagaaggtaccccagctgcgacattgaagtgtggtgcctacactatcgtctatgcacctataaaagaccaaacagatcccgcaccaagatatatctctggtgaagttacatctgtaacctttgaaaagagtgataatacagttaaaatcaaggttaacggtcaggatttcagcactctctctgctaattcaagtagtccaactgaaaatggcggatctgcgggtcaggcttcatcaagatcaagaagatcactctcagaggaaaccagtgaagctgctgcaaccgtcgatttgtttgcctttacccttgatggtggtaaaagaattgaagtggctgtaccaaacgtcgaagatgcatctaaaagagacaagtacagtttggttgcagacgataaacctttctataccggcgcaaacagcggcactaccaatggtgtctacaggttgaatgagaacggagacttggttgataaggacaacacagttcttttgaaggatgctggttcctctgcttttggactcagatacatcgttccttccgtttttgcaatctttgcagccttattcgtgttgtaaatttttttcaattaaattttaaaagtttaagagttttaagagtaattgcaatggaaatctttcgtgcgaattc

SEQ ID NO: 2 - protein, 324 aa, Artificial Sequence
  Description of Artificial Sequence/note = synthetic construct
MRLSLIIVLLSVIVSAVFSAPAVPLRGTLKDVPVEGSSSSSSSSSSSSSSSSSTSTVAPANKARTGEDAEGSQDSSGTEASGSQGSEEEGSEDDGQTSAASQPTTPAQSEGATTETIEATPKEECGTSFVMWFGEGTPAATLKCGAYTIVYAPIKDQTDPAPRYISGEVTSVTFEKSDNTVKIKVNGQDFSTLSANSSSPTENGGSAGQASSRSRRSLSEETSEAAATVDLFAFTLDGGKRIEVAVPNVEDASKRDKYSLVADDKPFYTGANSGTTNGVYRLNENGDLVDKDNTVLLKDAGSSAFGLRYIVPSVFAIFAALFVL

SEQ ID NO: 3 - protein, 17 aa, Artificial Sequence
  Description of Artificial Sequence/note = synthetic construct
ETDGAAAQVDLFAFQLD

SEQ ID NO: 4 - protein, 19 aa, Artificial Sequence
  Description of Artificial Sequence/note = synthetic construct
ETDEAAATVDLFAFTLDGG

SEQ ID NO: 5 - protein, 23 aa, Artificial Sequence
  VARIANT 3: Xaa = g or s
ETXEAAAGSVDLFAFGSLDGGKR

SEQ ID NO: 6 - protein, 10 aa, Artificial Sequence
  Description of Artificial Sequence/note = synthetic construct
IEVAVPNVED

SEQ ID NO: 7 - protein, 26 aa, Artificial Sequence
  Description of Artificial Sequence/note = synthetic construct
DKYSLVADDKPFYTGANSGTTNGVYR

SEQ ID NO: 8 - protein, 24 aa, Artificial Sequence
  Description of Artificial Sequence/note = synthetic construct
YSLVADDKPFYTGANSGTTNGVYR

SEQ ID NO: 9 - protein, 17 aa, Artificial Sequence
  Description of Artificial Sequence/note = synthetic construct
LNENGDLVDKDNTVLLK

SEQ ID NO: 10 - DNA, 29 bp, Artificial Sequence
  Description of Artificial Sequence/note = synthetic construct
cgcggatccttygcnttyacnctngaygg

SEQ ID NO: 11 - DNA, 30 bp, Artificial Sequence
  Description of Artificial Sequence/note = synthetic construct
gcggaattcnacngtrttrtcyttrtcnac

SEQ ID NO: 12 - protein, 7 aa, Artificial Sequence
  Description of Artificial Sequence/note = synthetic construct
FAFTLDG

SEQ ID NO: 13 - protein, 7 aa, Artificial Sequence
  Description of Artificial Sequence/note = synthetic construct
VDKDNTV

SEQ ID NO: 14 - DNA, 192 bp, Artificial Sequence
  Description of Artificial Sequence/note = synthetic construct
tttgcctttacccttgatggtggtaaaagaattgaagtggctgtaccaaacgtcgaagatgcatctaaaagagacaagtacagtttggttgcagacgataaacctttctataccggcgcaaacagcggcactaccaatggtgtctacaggttgaatgagaacggagacttggttgataaggacaacacagtt

SEQ ID NO: 15 - protein, 64 aa, Artificial Sequence
  Description of Artificial Sequence/note = synthetic construct
FAFTLDGGKRIEVAVPNVEDASKRDKYSLVADDKPFYTGANSGTTNGVYRLNENGDLVDKDNTV

SEQ ID NO: 16 - DNA, 33 bp, Artificial Sequence
  Description of Artificial Sequence/note = synthetic construct
ggtgtctacaggttgaatgagaacggagacttg

SEQ ID NO: 17 - DNA, 29 bp, Artificial Sequence
  Description of Artificial Sequence/note = synthetic construct
cgcggatccgaaaccagtgaagctgctgc

SEQ ID NO: 18 - DNA, 31 bp, Artificial Sequence
  Description of Artificial Sequence/note = synthetic construct
gcggaattcttaatccttcaaaagaactgtg

SEQ ID NO: 19 - protein, 79 aa, Artificial Sequence
  Description of Artificial Sequence/note = synthetic construct
ETSEAAATVDLFAFTLDGGKRIEVAVPNVEDASKRDKYSLVADDKPFYTGANSGTTNGVYRLNENGDLVDKDNTVLLKD

SEQ ID NO: 20 - DNA, 30 bp, Artificial Sequence
  Description of Artificial Sequence/note = synthetic construct
cgcggatccatgggttgttcatcatcaaag

SEQ ID NO: 21 - DNA, 29 bp, Artificial Sequence
  Description of Artificial Sequence/note = synthetic construct
gcggaattcattaggcatcagctggcttg

SEQ ID NO: 22 - protein, 148 aa, Artificial Sequence
  Description of Artificial Sequence/note = synthetic construct
MGNLKSCCSFADEHSLTSTQLVVGNGSGASETASNHPQEEVNDINTFNVKLIMQDRSKLDCEVVFDSTSISLSGDGKCRNIALDEIHQLLYSKEELSRVESSAGISDSDNCVAIHLKESGNCIPLFFNNSQDKERFVATANKFKPNFN